SEQUENCE LISTING (27 sequences)

SEQ ID NO: 1 - protein, 138 aa, synthetic construct
  REGION 1..138: note = aVEGF_Gly_Cys DARPin
SNAGSDLDKKLLEAARAGQDDEVRILMANGADVNARDSTGWTPLHLAAPWGHPEIVEVLLKNGADVNAADFQGWTPLHLAAAVGHLEIVEVLLKYGADVNAQDKFGKTAFDISIDNGNEDLAEILQKAAGGGSGGGSC

SEQ ID NO: 2 - protein, 117 aa, synthetic construct
  REGION 1..117: note = IL-15
SNANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 127 aa, synthetic construct
  REGION 1..127: note = Anti-VEGF VHH
MQVQLVESGGGLVQPGGSLRLSCAASGFAYSTYSMGWFRQAPGKEREAVATINSGTFRLWYTDSVKGRFTISRDNSKNTLYLQMNSLRPEDTAVYYCAARAWSPYSSTVDAGDFRYWGQGTLVTVSS

SEQ ID NO: 4 - protein, 122 aa, synthetic construct
  REGION 1..122: note = Anti-PD-1 VHH
SNAEVQLVESGGGLVQPGGSLRLSCAASGSIFSIHAMGWFRQAPGKEREFVAAITWSGGITYYEDSVKGRFTISRDNSKNTVYLQMNSLRPEDTAVYYCAADRAESSWYDYWGQGTLVTVSS

SEQ ID NO: 5 - protein, 136 aa, synthetic construct
  REGION 1..136: note = IL-2
SNAAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 6 - protein, 203 aa, synthetic construct
  REGION 1..203: note = IL-15Ra-IL-15 fusion
SNAGITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - protein, 248 aa, synthetic construct
  REGION 1..248: note = Anti-IL-1B scFv
SNAEIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVT

SEQ ID NO: 8 - protein, 146 aa, synthetic construct
  REGION 1..146: note = Anti-VEGF VHH
MQVQLVESGGGLVQPGGSLRLSCAASGFAYSTYSMGWFRQAPGKEREAVATINSGTFRLWYTDSVKGRFTISRDNSKNTLYLQMNSLRPEDTAVYYCAARAWSPYSSTVDAGDFRYWGQGTLVTVSSAEAAAKEAAAKEAAAKAGC

SEQ ID NO: 9 - protein, 141 aa, synthetic construct
  REGION 1..141: note = Anti-PD-1 VHH
SNAEVQLVESGGGLVQPGGSLRLSCAASGSIFSIHAMGWFRQAPGKEREFVAAITWSGGITYYEDSVKGRFTISRDNSKNTVYLQMNSLRPEDTAVYYCAADRAESSWYDYWGQGTLVTVSSAEAAAKEAAAKEAAAKAGC

SEQ ID NO: 10 - protein, 155 aa, synthetic construct
  REGION 1..155: note = IL-2
SNAAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTAEAAAKEAAAKEAAAKAGC

SEQ ID NO: 11 - protein, 137 aa, synthetic construct
  REGION 1..137: note = IL-15
SNANWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSAAEAAAKEAAAKEAAAKAGC

SEQ ID NO: 12 - protein, 223 aa, synthetic construct
  REGION 1..223: note = IL-15Ra-IL-15 fusion
SNAGITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSAAEAAAKEAAAKEAAAKAGC

SEQ ID NO: 13 - protein, 62 aa, synthetic construct
  REGION 1..62: note = Anti-TNFa Affibody
CGGGVDNKFNKEVGWAFGEIGALPNLNALQFRAFIISLWDDPSQSANLLAEAKKLNDAQAPK

SEQ ID NO: 14 - protein, 268 aa, synthetic construct
  REGION 1..268: note = Anti-IL-1B scFv
SNAEIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSSSPSTPPTPSPSTPPGGC

SEQ ID NO: 15 - protein, 144 aa, synthetic construct
  REGION 1..144: note = Anti-MCSP VHH
SNAEVQLQASGGGFVQPGGSLRLSCAASGTYSRITTMGWFRQAPGKEREFVSAISFASDNTPYYADSVKGRFTISRDNSKNTVYLQMNSLRAEDTATYYCAAWASRSTKAPMRYWGQGTQVTVSSAEAAAKEAAAKEAAAKAGC

SEQ ID NO: 16 - protein, 125 aa, synthetic construct
  REGION 1..125: note = Anti-MCSP VHH
SNAEVQLQASGGGFVQPGGSLRLSCAASGTYSRITTMGWFRQAPGKEREFVSAISFASDNTPYYADSVKGRFTISRDNSKNTVYLQMNSLRAEDTATYYCAAWASRSTKAPMRYWGQGTQVTVSS

SEQ ID NO: 17-20 - skipped (4 entries)

SEQ ID NO: 21 - protein, 19 aa, synthetic construct
AEAAAKEAAAKEAAAKAGC

SEQ ID NO: 22 - protein, 21 aa, synthetic construct
AEEEKRKAEEEKRKAEEEAGC

SEQ ID NO: 23 - protein, 28 aa, synthetic construct
AEEEKRKAEEEKRKAEEEKRKAEEEAGC

SEQ ID NO: 24 - protein, 20 aa, synthetic construct
AEEEEKKKKEEEEKKKKAGC

SEQ ID NO: 25 - protein, 14 aa, synthetic construct
AEAAAKEAAAKAGC

SEQ ID NO: 26 - protein, 16 aa, synthetic construct
PSRLEEELRRRLTEGC

SEQ ID NO: 27 - protein, 77 aa, synthetic construct
AEEEEKKKQQEEEAERLRRIQEEMEKERKRREEDEERRRKEEEERRMKLEMEAKRKQEEEERKKREDDEKRKKKAGC

(19) United States
(12) Patent Application Publication
(10) Pub. No.: US 2025/0213704 A1
(43) Pub. Date: Jul. 3, 2025

(54) METHOD OF TREATING A CANCER WITH A MULTIVALENT PEPTIDE CONJUGATE

(71) Applicant: Valitor, Inc, Berkeley, CA (US)

(72) Inventors: Wesley M. JACKSON, Berkeley, CA (US); Livia W. BRIER, Berkeley, CA (US); Amy A. TWITE, Berkeley, CA (US); Adam BARNEBEY, Berkeley, CA (US)

(21) Appl. No.: 18/848,753

(22) PCT Filed: Mar. 21, 2023
(86) PCT No.: PCT/US2023/064741

§ 371 Date: Sep. 19, 2024

Related U.S. Application Data
(60) Provisional application No. 63/321,971, filed on Mar. 21, 2022.

(51) Int. Cl. A61K 47/61; A61K 9/00

(52) U.S. Cl. CPC A61K 47/61; A61K 9/0019

(57) ABSTRACT

The present disclosure is directed to peptide-polymer conjugates, and their use in treating cancers, such as solid tumors.

### CROSS-REFERENCES TO RELATED APPLICATIONS

This application claims priority to U.S. Provisional Application No. 63/321,971, filed Mar. 21, 2022, which is incorporated herein in its entirety for all purposes.

### SEQUENCE LISTING

The material in the accompanying sequence listing is hereby incorporated by reference in its entirety. The accompanying file, named 2023-03-15 Sequence_Listing_ST26 052566-509001WO.xml was created on Mar. 15, 2023, and is 29.5 kilobytes in size.

### BACKGROUND OF THE INVENTION

Immunotherapeutic peptides have substantial utility in treating cancer patients. Cancer cells express neoantigens that can be recognized as foreign by the immune system, but tumors employ several mechanisms that block tumor immune recognition. Immune checkpoint inhibitors target negative immune signals that are upregulated by cancer cells to suppress antigen detection. Three drugs have been approved as of 2021 for clinical use against melanoma either as monotherapies or in combination and have improved the overall response rate for late stage melanoma patients. Combination checkpoint inhibitor therapies are more effective, but put patients at a higher risk of treatment-related adverse effects. Immune activating cytokines that stimulate proliferation of cytotoxic cell populations against tumor-specific antigens were clinically used as a treatment for malignant melanoma, but systemic administration of these cytokines can cause severe side effects such as vascular leakage and cytokine storms, rendering this therapy intolerable for most patients.

There are two routes of administration that are used to deliver a drug therapy to treat cancer: (1) systemic administration (i.e., oral, subcutaneous, and intravenous), or (2) intra-tumoral injection.

Systemic administration can often be performed easily, by the patient at home or in an outpatient infusion center, and thus re-dosing the patient may not be a challenge. However, for potent drug products, high systemic levels of the drugs required to maintain a therapeutic dose in the tumor site can also cause dangerous side effects elsewhere in the body, often causing secondary illnesses in the patient and requiring additional treatments. In addition, entire classes of potentially beneficial immunotherapies may not be available because systemic administration of these drugs can cause severe life-threatening side effects that make the therapy intolerable for most patients.

The advantage of intra-tumoral administration is that potent drugs can be delivered directly into the tumor. Because the drug has been administered locally to the target tissue, the dose delivered directly into the tumor can be lower than would be required to achieve the same therapeutic effect after systemic administration. However, intra-tumoral administration requires a professional to safely provide the injection. Thus, intra-tumoral injections can be more burdensome and costly to administer.

An additional challenge for administration directly to the primary tumor site is that the circulatory and lymphatic system can cause proteins and small molecules to be rapidly cleared from the injection site. As a result, drugs that are administered directly to the tumor are often cleared from the site and into the bloodstream where they can no longer act effectively on the diseased tissues. Drugs delivered by intra-tumoral injection may need to be re-dosed frequently to maintain efficacy, thus limiting their adoption in the clinic.

It would be useful to improve the intra-tumoral residence time of drugs to improve their efficacy and enable their adoption in the clinic. Particularly, it would be desirable to modify an existing drug to increase its residence time within the intra-tumoral space. By reducing the frequency of intra-tumoral administration of a drug, it may be possible to reduce the overall risk of complications associated with chronic administration of a drug. Prolonged intratumoral residence time with low systemic exposure could also enable more classes of immunotherapies to be safely used to treat patients. Increasing the duration of bioactivity may also yield enhanced therapeutic outcomes.

Additionally, a drug that exhibits a longer intra-tumoral residence time may be preferred by the patient compared to an alternate drug product that must be administered more frequently for an equivalent therapeutic function.

A conjugate comprising a biologically active polypeptide drug and a biocompatible polymer can exhibit an intra-tumoral half-life that is greater than the intra-tumoral half-life of the biologically active polypeptide when it is not conjugated to the biocompatible polymer. Increasing the half-life of the drug may have the effect of increasing the amount of time that the drug is above the concentration threshold required to generate an effective therapeutic response.

The increased half-life of the conjugate can confer certain advantages, including reduced burden on the patient; reduced number and/or frequency of administrations; increased safety; decreased incidence of infection; increased patient compliance; and increased efficacy. In addition, these conjugates may also enable broader use of certain classes of polypeptides for treatment of cancer that otherwise would be too risky to deliver to patients at higher systemic levels.

### BRIEF SUMMARY OF THE INVENTION

In some embodiments, a conjugate is the conjugate of Formula (I):

(X—Y)ₙ—Z (I),
wherein
each X is a peptide having a molecular weight of from about 5 kDa to about 150 kDa;
each Y is an organic linker having a molecular weight of from about 100 Da to about 500 Da;
Z is a biocompatible cellulose polymer having a molecular weight of from about 0.01 MDa to about 3 MDa; and
subscript n is an integer of from 5 to 500.

In some embodiments, a conjugate is the conjugate of Formula (I):

(X—Y)ₙ—Z (I),
wherein
each X is a peptide having a molecular weight of from about 5 kDa to about 150 kDa;
each Y is an organic linker having a molecular weight of from about 100 Da to about 500 Da;
Z is a biocompatible cellulose polymer having a molecular weight of from about 0.1 MDa to about 3 MDa; and
subscript n is an integer of from 5 to 500.

In some embodiments, a conjugate is the conjugate of Formula (Ia):

(X—Y)ₙ—Z (Ia),
wherein
X is a peptide having an amino acid sequence of SEQ ID NO: 1;
Y is an organic linker having the structure:

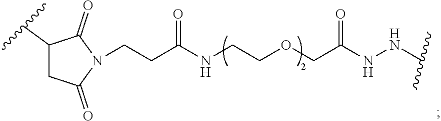

Z is a carboxymethyl cellulose polymer having a molecular weight of about 0.7 MDa; and subscript n is an integer of from about 10 to about 50.

In some embodiments, a pharmaceutical composition comprises a conjugate of the present invention and a pharmaceutically acceptable excipient.

In some embodiments, a method of treating a cancer in a human subject in need thereof comprises administering to the subject a therapeutically effective amount of a conjugate of the present invention or pharmaceutical composition thereof.

### BRIEF DESCRIPTION OF THE DRAWINGS

FIGS. 1A-1G show representative SDS-PAGE gels of conjugates. FIG. 1A: Conjugate 103. Column 1 shows the molecular weight ladder (kDa). Column 2 shows Conjugate 103 (5.0 μg); columns 3 and 4 show anti-PD-1 VHH (SEQ ID NO: 9) (0.5 μg and 0.75 μg, respectively). FIG. 1B: Conjugate 104. Column 1 shows the molecular weight ladder (kDa). Column 2 shows Conjugate 104 (5.0 μg); column 3 shows IL-2 (SEQ ID NO: 10) (0.5 μg). FIG. 1C: Conjugate 105. Column 1 shows the molecular weight ladder (kDa). Column 2 shows Conjugate 105 (5.0 μg); column 3 shows IL-15 (SEQ ID NO: 11) (0.75 μg). FIG. 1D: Conjugate 107. Column 1 shows anti-TNFα affibody (SEQ ID NO: 13). Column 2 shows Conjugate 107 (4 μg); column 3 shows the molecular weight ladder (kDa). FIG. 1E: Conjugate 108. Column 1 shows the molecular weight ladder (kDa). Column 2 shows Conjugate 108 (1.2 μg). FIG. 1F: Conjugate 101. Column 1 shows the molecular weight ladder (kDa); column 2 shows anti-VEGF DARPin (SEQ ID NO: 1) (5 μg); column 3 shows Conjugate 101 (5 μg). FIG. 1G: Conjugates 113, 114, and 115. Column 1 shows anti-VEGF VHH (SEQ ID NO: 8) (1.0 μg); column 2 shows Conjugate 113 (6 μg); column 3 shows Conjugate 114 (6 μg); column 4 shows Conjugate 115 (6 μg).

FIG. 2A-2C show results of intra-tumoral administration in xenografts in mice. FIG. 2A shows intra-tumoral half-lives of anti-VEGF DARPin antibodies (SEQ ID NO: 1; n=4) and conjugates made from anti-VEGF DARPin conjugated to carboxymethyl cellulose (CMC) with molecular weights (MW) of 700 kDa (n=4), and 90 kDa (n=5) injected into established SK-MEL-28 melanoma tumors in nude mice. The intra-tumoral half-lives were calculated using exponential decay fits of region of interest (ROI) radiant efficiency of infrared-tagged treatment peptides over 168 hours. The half-life of conjugates made using 700 kDa CMC was significantly longer than the unconjugated DARPin (** p<0.005, one-way ANOVA). FIG. 2B shows intra-tumoral half-lives of anti-VEGF DARPin antibodies (SEQ ID NO: 1; n=3) and conjugates made from anti-VEGF DARPin conjugated to CMC with a MW of 700 kDa (n=3), and hyaluronic acid with a MW of 860 kDa (n=3) injected into established MDA-MB-231 breast cancer tumors in nude mice. The intra-tumoral half-lives were calculated using exponential decay fits of ROI radiant efficiency of infrared-tagged treatment peptides over 264 hours. The half-life of conjugates made using 700 kDa CMC was significantly longer than the unconjugated DARPin and with the conjugate having hyaluronic acid biopolymer (* p<0.05, one-way ANOVA). FIG. 2C shows representative images of anti-VEGF DARPin antibodies and conjugates made from anti-VEGF DARPin conjugated to CMC with MWs of 700 kDa injected into established SK-MEL-28 melanoma tumors in nude mice 7 days after injection.

### DETAILED DESCRIPTION OF THE INVENTION

### I. Definitions

“About” when referring to a value includes the stated value+/−10% of the stated value. For example, about 50% includes a range of from 45% to 55%, while about 20 molar equivalents includes a range of from 18 to 22 molar equivalents. Accordingly, when referring to a range, “about” refers to each of the stated values+/−10% of the stated value of each end of the range. For instance, a ratio of from about 1 to about 3 (weight/weight) includes a range of from 0.9 to 3.3.

“Alkyl” is a linear or branched saturated monovalent or divalent hydrocarbon. For example, an alkyl group can have 1 to 10 carbon atoms (i.e., C₁₋₁₀ alkyl) or 1 to 8 carbon atoms (i.e., C₁₋₈ alkyl) or 1 to 6 carbon atoms (i.e., C₁₋₆ alkyl) or 1 to 4 carbon atoms (i.e., (C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

“Cycloalkyl” refers to a single saturated or partially unsaturated all carbon ring having 3 to 20 annular carbon atoms (i.e., C₃₋₂₀ cycloalkyl), for example from 3 to 12 annular atoms, for example from 3 to 10 annular atoms, or 3 to 8 annular atoms, or 3 to 6 annular atoms, or 3 to 5 annular atoms, or 3 to 4 annular atoms. The term “cycloalkyl” also includes multiple condensed, saturated and partially unsaturated all carbon ring systems (e.g., ring systems comprising 2, 3 or 4 carbocyclic rings). Accordingly, cycloalkyl includes multicyclic carbocycles such as a bicyclic carbocycles (e.g., bicyclic carbocycles having about 6 to 12 annular carbon atoms such as bicyclo[3.1.0]hexane and bicyclo[2.1.1]hexane), and polycyclic carbocycles (e.g. tricyclic and tetracyclic carbocycles with up to about 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

“Antibody” as used herein refers to a polypeptide encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Antibodies are representative of a wide variety of receptors including hormone receptors, drug targets such as peripheral benzodiazepine receptor, and carrier proteins. Representative antibodies include, but are not limited to monoclonal IgG antibodies, IgG antibody fragments, single chain scFv antibodies, single-domain heavy-chain VHH antibodies, or engineered antibody-like scaffolds such as adnectins, affibodies, anticalins, DARPins, and engineered Kunitz-type inhibitors. Other examples also include receptor decoys of immunomodulatory cytokines such as Tumor Necrosis Factor-α and IL-1β, IL-6, or interferon-γ.

“Biocompatible cellulose polymer” as used herein refers to a cellulose-based polymer compatible with in vivo administration. Biocompatible cellulose polymers include, but are not limited to methyl cellulose (MC), ethyl cellulose (EC), hydroxyethyl cellulose (HEC), hydroxypropyl cellulose (HPC), hydroxypropylmethyl cellulose (HPMC), carboxymethyl cellulose (CMC) and sodium carboxymethyl cellulose (NaCMC).

“Immune cell function” includes, for example, modulation of an immune response. The modulation can be immunosuppressive or immunostimulatory. Examples of immune responses can include, but are not limited to a humoral immune response, a cell-mediate immune response, or an inflammatory response.

“Inhibition”, “inhibits” and “inhibitor” as used herein refer to a compound that prohibits or a method of prohibiting, a specific action or function.

“Modulate” as used herein refers to the ability of a compound to increase or decrease the function, or activity, of the associated activity (e.g., immune cell function).

“Organic linker” as used herein refers to a chemical moiety that directly or indirectly covalently links the peptide to the polymer. Organic linkers useful in the present invention can be about 100 Da to 500 Da. The types of organic linkers of the present invention include, but are not limited to, imides, amides, amines, esters, carbamates, ureas, thioethers, thiocarbamates, thiocarbonate and thioureas. One of skill in the art will appreciate that other types of organic linkers are useful in the present invention.

“Thiol” refers to the —SH functional group.

“Thiol reactive group” refers to a group capable of reacting with a thiol to form a covalent bond to the sulfur atom. Representative thiol reactive groups include, but are not limited to, thiol, TNB-thiol, haloacetyl, aziridine, acryloyl, vinylsulfone, APN (3-arylpropionitrile), maleimide and pyridyl disulfide. Reaction of the thiol reactive group with a thiol can form a disulfide or a thioether.

“Peptide,” “polypeptide,” and “protein” are used interchangeably herein, and refer to naturally occurring and synthetic amino acids of any length, as well as amino acid analogs and amino acid mimetics that function in a manner similar to the naturally occurring amino acids. The term “polypeptide” includes fusion proteins, including, but not limited to, fusion proteins with a heterologous amino acid sequence, fusions with heterologous and homologous leader sequences, with or without N-terminal methionine residues; immunologically tagged proteins; and the like. The term “polypeptide” includes post-translationally modified polypeptides.

“VHH” as used herein refers to a single-domain heavy chain antibody.

“DARPin” refers to a designed ankyrin repeat protein, which is a genetically engineered antibody mimetic protein that can exhibit highly specific and high-affinity target protein binding.

An “alpha-helix” or “α-helix” is a common motif in the secondary structure of proteins and is a right hand-helix conformation in which every backbone N—H group hydrogen bonds to the backbone C═O group of the amino acid located four residues earlier along the protein sequence. The alpha-helix is also known as a classic Pauling-Corey-Branson α-helix, or 3.6₁₃-helix, which denotes the average number of residues per helical turn (3.6) with 13 atoms being involved in the ring formed by the hydrogen bond. Peptides that contain an alpha-helix is said to be alpha-helical. Such peptides may be partly or entirely alpha-helical. As understood in the art, an alpha-helix has at least four amino acid residues. In some embodiments, an alpha-helix has from 4 to 40 amino acids.

Provided are also pharmaceutically acceptable salts of the peptides or conjugates described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

“Pharmaceutical composition” as used herein refers to a product comprising the specified ingredients in the specified amounts, as well as any product, which results, directly or indirectly, from combination of the specified ingredients in the specified amounts. The pharmaceutical composition is generally safe for biological use.

“Pharmaceutically acceptable carrier” and “pharmaceutically acceptable excipient” as used herein refers to a substance that aids the administration of an active agent to an absorption by a subject. Pharmaceutical carrier and/or excipient useful in the present invention include, but are not limited to, binders, fillers, disintegrants, lubricants, coatings, sweeteners, flavors and colors. One of skill in the art will recognize that other pharmaceutical carriers and/or excipients are useful in the present invention.

“Polymer molecular weight” as used herein refers to the molecular weight of the polymer.

“Treatment” or “treat” or “treating” as used herein refers to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In some embodiments, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

“Therapeutically effective amount” or “effective amount” as used herein refers to an amount that is effective to elicit the desired biological or medical response, including the amount of a compound that, when administered to a subject for treating a disease, is sufficient to effect such treatment for the disease. The effective amount can vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Administering” refers to intra-tumoral administration to the subject. The administration can be carried out according to a schedule specifying frequency of administration, dose for administration, and other factors.

“Subject” refers to animals such as mammals, including, but not limited to, primates (e.g., humans), cows, sheep, goats, horses, dogs, cats, rabbits, rats, mice and the like. In some embodiments, the subject is a human.

### II. Peptide-Polymer Conjugates

In some embodiments, a conjugate of the present invention comprises biocompatible cellulose polymers and a plurality of peptides that possess a potency greater than a similar concentration of the unconjugated peptide, where the peptides are covalently linked to the polymer via an organic linker. In some embodiments, the conjugate is a conjugate of Formula I:

(X—Y)ₙ—Z (I),

wherein each X is a peptide having a molecular weight of from about 5 kDa to about 150 kDa; each Y is an organic linker having a molecular weight of from about 100 Da to about 500 Da; Z is a biocompatible cellulose polymer having a molecular weight of from about 0.01 MDa to about 3 MDa; and subscript n is an integer of from 5 to 500. In some embodiments, the conjugate is a conjugate of Formula I:

(X—Y)ₙ—Z (I),

wherein each X is a peptide having a molecular weight of from about 5 kDa to about 150 kDa; each Y is an organic linker having a molecular weight of from about 100 Da to about 500 Da; Z is a biocompatible cellulose polymer having a molecular weight of from about 0.1 MDa to about 3 MDa; and subscript n is an integer of from 5 to 500.

### Peptides

Suitable peptides of the present invention include peptides that selectively bind a protein implicated the function and/or growth of a cancer. In some embodiments, the peptide selectively binds B cell maturation antigen (BCMA), CD19, CD20, CD40, CD44, CD47, CD80, cytotoxic T-lymphocyte-associated antigen 4 (CTLA4), epidermal growth factor receptor (EGFR), epithelial cell adhesion molecule (EpCAM), glucose transport protein (GTR), human epithelial growth factor receptor 2 (HER2), interleukin-5 (IL-5), interleukin-18 (IL-18), melanoma-associated chondroitin sulfate proteoglycan (MCSP), mesothelin, programmed cell death protein 1 (PD-1), programmed death-ligand 1 (PD-L1), programmed cell death 1 ligand 2 (PD-L2), tumor necrosis factor-alpha (TNF-α), unique long-16 binding protein 2 (ULBP2), unique long-16 binding protein 5 (ULBP5), unique long-16 binding protein 6 (ULBP6), or vascular endothelial growth factor receptor (VEGFR). In some embodiments, the peptide selectively binds epidermal growth factor receptor (EGFR), tumor necrosis factor-alpha (TNF-αt), or vascular endothelial growth factor receptor (VEGFR). In some embodiments, the peptide selectively binds interleukin-2 (IL-2) or interleukin-15 (IL-15). In some embodiments, the peptide selectively binds vascular endothelial growth factor receptor (VEGFR). In some embodiments, the peptide selectively binds interleukin-15 (IL-15).

In some embodiments, the peptide modulates the activity of inmmne cell function. Examples of peptides that are designed to attenuate immune cell function include immune activating cytokines such as interleukin (IL)-15, IL-2, and IL-12. Other examples include selective antibody inhibitors immune checkpoints, receptor decoys of tumor necrosis factor-α, IL-1β, IL-6, and interferon-γ.

In some embodiments, the peptide is a monoclonal IgG, an IgG fragment, single chain scFv, single-domain heavy-chain VHH, adnectin, affibody, anticalin, DARPin, or an engineered Kunitz-type inhibitor.

Peptides suitable in the present invention are those having a molecular weight of at least about 2 kDa, and exhibit tertiary structure. Representative peptides include, but are not limited to, polypeptides, one or more aptamers, avimer scaffolds based on human A domain scaffolds, diabodies, camelids, shark IgNAR antibodies, fibronectin type III scaffolds with modified specificities, antibodies, antibody fragments, proteins, peptides, polypeptides.

In some embodiments, the peptide is a therapeutic protein. Numerous therapeutic proteins are disclosed throughout the application such as, and without limitation, erythropoietin, granulocyte colony stimulating factor (G-CSF), GM-CSF, interferon alpha, interferon beta, human growth hormone, and imiglucerase.

Peptides that are suitable for inclusion in a conjugate, for use in a method of the present disclosure, include, but are not limited to, neuroprotective polypeptides (e.g., GDNF, CNTF, NT4, NGF, and NTN); anti-angiogenic polypeptides (e.g., a soluble vascular endothelial growth factor (VEGF) receptor; a VEGF-binding antibody; a VEGF-binding antibody fragment (e.g., a single chain anti-VEGF antibody); endostatin; tumstatin; angiostatin; a soluble Flt polypeptide (Lai et al. (2005) Mol. Ther. 12:659); an Fc fusion protein comprising a soluble Flt polypeptide (see, e.g., Pechan et al. (2009) Gene Ther. 16:10); pigment epithelium-derived factor (PEDF); a soluble Tie-2 receptor; etc.); tissue inhibitor of metalloproteinases-3 (TIMP-3); a light-responsive opsin, e.g., a rhodopsin; anti-apoptotic polypeptides (e.g., Bcl-2, Bcl-Xl); and the like. Suitable polypeptides include, but are not limited to, glial derived neurotrophic factor (GDNF); fibroblast growth factor 2; neurturin (NTN); ciliary neurotrophic factor (CNTF); nerve growth factor (NGF); neurotrophin-4 (NT4); brain derived neurotrophic factor (BDNF); epidermal growth factor; rhodopsin; X-linked inhibitor of apoptosis; and Sonic hedgehog.

Peptides that are suitable for inclusion in a conjugate, for use in a method of the present disclosure, include, but are not limited to, a soluble vascular endothelial growth factor (VEGF) receptor; angiostatin, endostatin; vasostatin; retinal pigment epithelium-specific protein 65 kDa (RPE65); and compstatin. In some embodiments, the biologically active polypeptide is a soluble fins-like tyrosine kinase-1 (sFlt-1) polypeptide. In some embodiments, the biologically active polypeptide is a single-domain camelid (VHH) anti-VEGF antibody (VHH anti-VEGF antibody). In some embodiments, the biologically active polypeptide is a single chain Fv anti-VEGF antibody (scFv anti-VEGF antibody). In some embodiments, the peptide is an adnectin, an affibody, an anticalin, a DARPin, a Kunitz-type inhibitor, or a receptor decoy.

Peptides that are suitable for inclusion in a conjugate, for use in a method of the present disclosure, include an antibody. Suitable antibodies include, e.g., an antibody specific for VEGF; an antibody specific for tumor necrosis factor-alpha (TNF-α); and the like.

In some embodiments, the peptide can be selected from specifically identified protein or peptide agents, including, but not limited to: Aβ, agalsidase, alefacept, alkaline phosphatase, aspariginase, amdoxovir (DAPD), antide, becaplermin, botulinum toxin including types A and B and lower molecular weight compounds with botulinum toxin activity, calcitonins, cyanovirin, denileukin diftitox, erythropoietin (EPO), EPO agonists, domase alpha, erythropoiesis stimulating protein (NESP), coagulation factors such as Factor V, Factor VII, Factor VIIa, Factor VIII, Factor IX, Factor X, Factor XII, Factor XIII, von Willebrand factor; ceredase, cerezyme, alpha-glucosidase, N-Acetylgalactosamine-6-sulfate sulfatase, collagen, cyclosporin, alpha defensins, beta defensins, desmopressin, exendin-4, cytokines, cytokine receptors, granulocyte colony stimulating factor (G-CSF), thrombopoietin (TPO), alpha-1 proteinase inhibitor, elcatonin, granulocyte macrophage colony stimulating factor (GM-CSF), fibrinogen, filgrastim, growth hormones human growth hormone (hGH), somatropin, growth hormone releasing hormone (GHRH), GRO-beta, GRO-beta antibody, bone morphogenic proteins such as bone morphogenic protein-2, bone morphogenic protein-6, parathyroid hormone, parathyroid hormone related peptide, OP-1; acidic fibroblast growth factor, basic fibroblast growth factor, Fibroblast Growth Factor 21, CD-40 ligand, heparin, human serum albumin, low molecular weight heparin (LMWH), interferon alpha, interferon beta, interferon gamma, interferon omega, interferon tau, consensus interferon, human lysyl oxidase-like-2 (LOXL2); interleukins and interleukin receptors such as interleukin-1 receptor, interleukin-2, interleukin-2 fusion proteins, interleukin-1 receptor antagonist, interleukin-3, interleukin-4, interleukin-4 receptor, interleukin-6, interleukin-8, interleukin-12, interleukin-15, interleukin-17, interleukin-21, interleukin-23, p40, interleukin-13 receptor, interleukin-17 receptor; lactoferrin and lactoferrin fragments, luteinizing hormone releasing hormone (LHRH), insulin, pro-insulin, insulin analogues, leptin, ghrelin, amylin, C-peptide, somatostatin, somatostatin analogs including octreotide, vasopressin, follicle stimulating hormone (FSH), imiglucerase, influenza vaccine, insulin-like growth factor (IGF), insulintropin, macrophage colony stimulating factor (M-CSF), plasminogen activators such as alteplase, urokinase, reteplase, streptokinase, pamiteplase, lanoteplase, and teneteplase; nerve growth factor (NGF), osteoprotegerin, platelet-derived growth factor, tissue growth factors, transforming growth factor-1, vascular endothelial growth factor, leukemia inhibiting factor, keratinocyte growth factor (KGF), glial growth factor (GGF), T Cell receptors, CD molecules/antigens, tumor necrosis factor (TNF) (e.g., TNF-α and TNF-β), TNF receptors (e.g., TNF-α receptor and TNF-β receptor), CTLA4, CTLA4 receptor, monocyte chemoattractant protein-1, endothelial growth factors, parathyroid hormone (PTH), glucagon-like peptide, somatotropin, thymosin alpha 1, rasburicase, thymosin alpha 1 IIb/IIIa inhibitor, thymosin beta 10, thymosin beta 9, thymosin beta 4, alpha-1 antitrypsin, phosphodiesterase (PDE) compounds, VLA-4 (very late antigen-4), VLA-4 inhibitors, bisphosphonates, respiratory syncytial virus antibody, cystic fibrosis transmembrane regulator (CFTR) gene, deoxyribonuclease (Dnase), bactericidal/permeability increasing protein (BPI), and anti-CMV antibody. Exemplary monoclonal antibodies include etanercept (a dimeric fusion protein consisting of the extracellular ligand-binding portion of the human 75 kD TNF receptor linked to the Fc portion of IgG1), abciximab, adalimumab, afelimomab, alemtuzumab, antibody to B-lymphocyte, atlizumab, basiliximab, bevacizumab, biciromab, bertilimumab, CDP-484, CDP-571, CDP-791, CDP-860, CDP-870, cetuximab, clenoliximab, daclizumab, eculizumab, edrecolomab, efalizumab, epratuzumab, fontolizumab, gavilimomab, gemtuzumab ozogamicin, ibritumomab tiuxetan, infliximab, inolimomab, keliximab, labetuzumab, lerdelimumab, olizumab, radiolabeled lym-1, metelimumab, mepolizumab, mitumomab, muromonad-CD3, nebacumab, natalizumab, odulimomab, omalizumab, oregovomab, palivizumab, pemtumomab, pexelizumab, rhuMAb-VEGF, rituximab, satumomab pendetide, sevirumab, siplizumab, tositumomab, I¹³¹-tositumomab, trastuzumab, tuvirumab, visilizumab, and fragments and mimetics thereof.

Suitable antibodies include, but are not limited to, adalimumab, alemtuzumab, basiliximab, belimumab, bevacizumab, briakinumab, brodalumab, canakinumab, certolizumab, claakizumab, daclizumab, denosumab, efalizumab, epratuzumab, etaracizumab, fezakinumab, figitumumab, fontolizumab, gevokizumab, gotimumab, infliximab, namilumab, namilumab, natalizumab, neutrazumab, nextomab, ocaratuzumab, ofatumumab, olokizumab, pateclizumab, priliximab, ranibizumab, rituximab, secukinumab, sirukumab, sonepcizumab, tabalumab, tocilizumab, toralizumab, ustekinumab, vapaliximab, vedolizumab, veltuzumab, visilizumab, vorsetuzumab, and ziralimumab.

In some embodiments, the peptide is a fusion protein. For example, and without limitation, the peptide can be an immunoglobulin or portion of an immunoglobulin fused to one or more certain useful peptide sequences. For example, the peptide may contain an antibody Fc fragment. In one embodiment, the peptide is a CTLA4 fusion protein. For example, the peptide can be an Fc-CTLA4 fusion protein. In another embodiment, the peptide is a Factor VIII fusion protein. For example, the peptide can be an Fc-Factor VIII fusion protein.

In some embodiments, the peptide is a human protein or human polypeptide, for example, a heterologously produced human protein or human polypeptide. Numerous proteins and polypeptides are disclosed herein for which there is a corresponding human form (i.e., the protein or peptide is normally produced in human cells in the human body). Therefore, in one embodiment, the peptide is the human form of each of the proteins and polypeptides disclosed herein for which there is a human form. Examples of such human proteins include, without limitation, human antibodies, human enzymes, human hormones and human cytokines such as granulocyte colony stimulation factor, granulocyte macrophage colony stimulation factor, interferons (e.g., alpha interferons and beta interferons), human growth hormone and erythropoietin.

Other examples of therapeutic proteins include, without limitation, factor VIII, b-domain deleted factor VIII, factor VIIa, factor IX, anticoagulants; hirudin, alteplase, tpa, reteplase, tpa, tpa-3 of 5 domains deleted, insulin, insulin lispro, insulin aspart, insulin glargine, long-acting insulin analogs, HGH, glucagons, tsh, follitropin-beta, fsh, gm-csf, pdgh, IFN alpha2, IFN alpha2a, IFN alpha2b, IFN-apha1, consensus IFN, IFN-beta, IFN-beta 1b. IFN-beta 1a, IFN-gamma (e.g., 1 and 2), IFN-lambda, IFN-delta, IL-2, IL-11, hbsag, ospa, murine mab directed against t-lymphocyte antigen, murine mab directed against TAG-72, tumor-associated glycoprotein, fab fragments derived from chimeric mab directed against platelet surface receptor gpII(b)/III(a), murine mab fragment directed against tumor-associated antigen cal 25, murine mab fragment directed against human carcinoembryonic antigen, cea, murine mab fragment directed against human cardiac myosin, murine mab fragment directed against tumor surface antigen psma, murine mab fragments (fab/fab2 mix) directed against hmw-maa, murine mab fragment (fab) directed against carcinoma-associated antigen, mab fragments (fab) directed against nca 90, a surface granulocyte nonspecific cross reacting antigen, chimeric mab directed against cd20 antigen found on surface of b lymphocytes, humanized mab directed against the alpha chain of the i12 receptor, chimeric mab directed against the alpha chain of the i12 receptor, chimeric mab directed against tnf-alpha, humanized mab directed against an epitope on the surface of respiratory synctial virus, humanized mab directed against her 2, human epidermal growth factor receptor 2, human mab directed against cytokeratin tumor-associated antigen anti-ctla4, chimeric mab directed against cd 20 surface antigen of b lymphocytes dornase-alpha dnase, beta glucocerebrosidase, tnf-alpha, il-2-diptheria toxin fusion protein, tnfr-lgg fragment fusion protein laronidase, dnaases, alefacept, darbepoetin alpha (colony stimulating factor), tositumomab, murine mab, alemtuzumab, rasburicase, agalsidase beta, teriparatide, parathyroid hormone derivatives, adalimumab (lggl), anakinra, biological modifier, nesiritide, human b-type natriuretic peptide (hbnp), colony stimulating factors, pegvisomant, human growth hormone receptor antagonist, recombinant activated protein c, omalizumab, immunoglobulin e (lge) blocker, lbritumomab tiuxetan, ACTH, glucagon, somatostatin, somatotropin, thymosin, parathyroid hormone, pigmentary hormones, somatomedin, erythropoietin, luteinizing hormone, chorionic gonadotropin, hypothalmic releasing factors, etanercept, antidiuretic hormones, prolactin and thyroid stimulating hormone. And any of these can be modified to have a site-specific conjugation point (a N-terminus, or C-terminus, or other location) using natural (for example, a serine to cysteine substitution) (for example, formylaldehyde per method of Redwood Biosciences) or non-natural amino acid.

Examples of therapeutic antibodies (or their respective scFv or Fab fragments) useful in the present invention include, but are not limited to, Anti-TNF inhibitors such as the TNF receptor decoy etanercept and the monoclonal antibodies adalimumab, infliximab, golimumab, and certolizumab, the IL-6 monoclonal antibody inhibitor siltuximab, the IL-17 monoclonal antibody inhibitors secukinumab and ixekizumab, the IL-12/23 monoclonal antibody inhibitor ustekinumab, integrin receptor antagonists such as the monoclonal antibody inhibitors natalizumab and etrolizumab, the CLTA receptor antagonist abatacept, the IL-13 monoclonal antibody inhibitor tralokinumab, chemokine inhibitors such as the monoclonal antibodies eldelumab and bertilumab, and IL-1 inhibitors such as the receptor decoy rilonacept and the such as the monoclonal antibody canakinumab.

Other examples of therapeutic antibodies (or their respective scFv or Fab fragments) useful in the present invention include, but are not limited, to HERCEPTIN™ (Trastuzumab) (Genentech, CA) which is a humanized anti-HER2 monoclonal antibody for the treatment of patients with metastatic breast cancer; REOPRO™ (abciximab) (Centocor) which is an anti-glycoprotein IIb/IIIc receptor on the platelets for the prevention of clot formation; ZENAPAX™ (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath; Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primate anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (CS) antibody (Alexion Pharm); D2E7 is a humanized anti-TNF-α antibody (CATIBASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β07 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); CAT-152, a human anti-TGF-β.sub.2 antibody (Cambridge Ab Tech); Cetuximab (BMS) is a monoclonal anti-EGF receptor (EGFr) antibody; Bevacizuma (Genentech) is an anti-VEGF human monoclonal antibody; Infliximab (Centocore, JJ) is a chimeric (mouse and human) monoclonal antibody used to treat autoimmune disorders; Gemtuzumab ozogamicin (Wyeth) is a monoclonal antibody used for chemotherapy; and Ranibizumab (Genentech) is a chimeric (mouse and human) monoclonal antibody used to treat macular degeneration.

Proteins and peptides disclosed herein can be produced by any useful method including production by in vitro synthesis and by production in biological systems. Typical examples of in vitro synthesis methods which are well known in the art include solid-phase synthesis (“SPPS”) and solid-phase fragment condensation (“SPFC”). Biological systems used for the production of proteins are also well known in the art. Bacteria (e.g., E. coli and Bacillus sp.), yeast (e.g., Saccharomyces cerevisiae and Pichia pastoris), and tobacco leaves (via tobacco mosaic virus) are widely used for the production of heterologous proteins. In addition, heterologous gene expression for the production of peptides for use as disclosed herein can be accomplished using animal cell lines such as mammalian cell lines (e.g., CHO cells). In one particularly useful embodiment, the peptides are produced in transgenic or cloned animals such as cows, sheep, goats and birds (e.g., chicken, quail, ducks and turkey), each as is understood in the art. See, for example, U.S. Pat. No. 6,781,030, the disclosure of which is incorporated in its entirety herein by reference.

Protein or polypeptides useful in the present invention may also comprise non-naturally occurring amino acids in addition to the common naturally occurring amino acids found in proteins and polypeptides. In addition to being present for the purpose of altering the properties of a polypeptide or protein, non-naturally occurring amino acids can be introduced to provide a functional group that can be used to link the protein or polypeptide directly to the random copolymer. Furthermore, naturally occurring amino acids, e.g., cysteine, tyrosine, tryptophan can be used in this way.

Non-naturally occurring amino acids can be introduced into proteins and peptides by a variety of means. Some of the techniques for the introduction of non-natural amino acids are discussed in U.S. Pat. No. 5,162,218, the disclosure of which is incorporated in its entirety herein by reference. First, non-naturally occurring amino acids can be introduced by chemical modification of a polypeptide or protein on the amino acid side chain or at either the amino terminus or the carboxyl terminus. Non-limiting examples of chemical modification of a protein or peptide might be methylation by agents such as diazomethane, or the introduction of acetylation at an amino group present in lysine's side chain or at the amino terminus of a peptide or protein. Another example of the protein/polypeptide amino group modification to prepare a non-natural amino acid is the use of methyl 3-mercaptopropionimidate ester or 2-iminothiolane to introduce a thiol (sulfhydryl, —SH) bearing functionality linked to positions in a protein or polypeptide bearing a primary amine. Once introduced, such groups can be employed to form a covalent linkage to the protein or polypeptide.

Second, non-naturally occurring amino acids can be introduced into proteins and polypeptides during chemical synthesis. Synthetic methods are typically utilized for preparing polypeptides having fewer than about 200 amino acids, usually having fewer than about 150 amino acids, and more usually having 100 or fewer amino acids. Shorter proteins or polypeptides having less than about 75 or less than about 50 amino acids can be prepared by chemical synthesis.

The synthetic preparation methods that are particularly convenient for allowing the insertion of non-natural amino acids at a desired location are known in the art. Suitable synthetic polypeptide preparation methods can be based on Merrifield solid-phase synthesis methods where amino acids are sequentially added to a growing chain (Merrifield (1963) J. Am. Chem. Soc. 85:2149-2156). Automated systems for synthesizing polypeptides by such techniques are now commercially available from suppliers such as Applied Biosystems, Inc., Foster City, Calif. 94404; New Brunswick Scientific, Edison, N.J. 08818; and Pharmacia, Inc., Biotechnology Group, Piscataway, N.J. 08854.

Examples of non-naturally occurring amino acids that can be introduced during chemical synthesis of polypeptides include, but are not limited to: D-amino acids and mixtures of D and L-forms of the 20 naturally occurring amino acids, N-formyl glycine, ornithine, norleucine, hydroxyproline, beta-alanine, hydroxyvaline, norvaline, phenylglycine, cyclohexylalanine, t-butylglycine (t-leucine, 2-amino-3,3-dimethylbutanoic acid), hydroxy-t-butylglycine, amino butyric acid, cycloleucine, 4-hydroxyproline, pyroglutamic acid (5-oxoproline), azetidine carboxylic acid, pipecolinic acid, indoline-2-carboxylic acid, tetrahydro-3-isoquinoline carboxylic acid, 2,4-diaminobutyricacid, 2,6-diaminopimelic acid, 2,4-diaminobutyricacid, 2,6-diaminopimelicacid, 2,3-diaminopropionicacid, 5-hydroxylysine, neuraminic acid, and 3,5-diiodotyrosine.

Third, non-naturally occurring amino acids can be introduced through biological synthesis in vivo or in vitro by insertion of a non-sense codon (e.g., an amber or ocher codon) in a DNA sequence (e.g., the gene) encoding the polypeptide at the codon corresponding to the position where the non-natural amino acid is to be inserted. Such techniques are discussed for example in U.S. Pat. Nos. 5,162,218 and 6,964,859, the disclosures of which are incorporated in their entirety herein by reference. A variety of methods can be used to insert the mutant codon including oligonucleotide-directed mutagenesis. The altered sequence is subsequently transcribed and translated, in vivo or in vitro in a system which provides a suppressor tRNA, directed against the nonsense codon that has been chemically or enzymatically acylated with the desired non-naturally occurring amino acid. The synthetic amino acid will be inserted at the location corresponding to the nonsense codon. For the preparation of larger and/or glycosylated polypeptides, recombinant preparation techniques of this type are usually preferred. Among the amino acids that can be introduced in this fashion are: formyl glycine, fluoroalanine, 2-Amino-3-mercapto-3-methylbutanoic acid, homocysteine, homoarginine and the like. Other similar approaches to obtain non-natural amino acids in a protein include methionine substitution methods.

Where non-naturally occurring amino acids have a functionality that is susceptible to selective modification, they are particularly useful for forming a covalent linkage to the protein or polypeptide. Circumstances where a functionality is susceptible to selective modification include those where the functionality is unique or where other functionalities that might react under the conditions of interest are hindered either stereochemically or otherwise.

Other antibodies, such as single domain antibodies are useful in the present invention. A single domain antibody (sdAb, called Nanobody by Ablynx) is an antibody fragment consisting of a single monomeric variable antibody domain. Like a whole antibody, the sdAb is able to bind selectively to a specific antigen. With a molecular weight of only 12-15 kDa, single domain antibodies are much smaller than common whole antibodies (150-160 kDa). A single domain antibody is a peptide chain of about 110 amino acids in length, comprising one variable domain (VH) of a heavy chain antibody, or of a common IgG.

Unlike whole antibodies, single domain antibody (sdAbs) such as VHH do not show complement system triggered cytotoxicity because they lack an Fc region. Camelid and fish derived sdAbs are able to bind to hidden antigens that are not accessible to whole antibodies, for example to the active sites of enzymes.

A sdAb can be obtained by immunization of dromedaries, camels, llamas, alpacas or sharks with the desired antigen and subsequent isolation of the mRNA coding for heavy chain antibodies. Alternatively they can be made by screening synthetic libraries. Camelids are members of the biological family Camelidae, the only living family in the suborder Tylopoda. Camels, dromedaries, Bactrian Camels, llamas, alpacas, vicunas, and guanacos are in this group.

Peptides useful in the present invention also include, but are not limited to, a macrocyclic peptide, a cyclotide, an LDL receptor A-domain, a soluble receptor, an enzyme, a peptide multimer, a domain multimer, an antibody fragment multimer, and a fusion protein.

In some embodiments, the peptide modulates the activity of immune cell function. In some embodiments, the peptide inhibits tumor necrosis factor-α, interleukin-1β, interleukin-6, or interferon-γ. In some embodiments, the peptide inhibits tumor necrosis factor-α. In some embodiments, the peptide is a monoclonal IgG antibody, an IgG antibody fragment, a single-chain variable region antibody, a single-domain heavy chain antibody, an adnectin, an affibody, an anticalin, a DARPin, a Kunitz-type inhibitor, or a receptor decoy.

In some embodiments, the peptide has an amino acid sequence according to any one of SEQ ID NOs: 1 to 16. In some embodiments, the peptide has an amino acid sequence of SEQ ID NO: 1. In some embodiments, the peptide has an amino acid sequence of SEQ ID NO: 2. In some embodiments, the peptide has an amino acid sequence of SEQ ID NO: 3.

### Organic Linkers

The organic linkers of the present invention are covalent linkers. In some embodiments, the organic linker is a hydrophilic organic linker. The organic linkers can include naturally occurring molecules like nucleic acid bases, dimers, and oligomers, carbohydrate monomers or oligosaccharides of a variety of compositions, dextrans, dipeptides or oligopeptides. Other organic linkers can include, but are not limited to, ethylene glycol dimers, trimers, oligomers and polymers, as well as polyvinyl alcohol, polyvinyl acetate, polyacrylate, peptoids, D- or artificial amino acid containing peptides, polymer brushes, polyelectrolyte brushes, synthetic carbohydrate mono and oligomers, cleavable linkers, or any combination of the above such as nucleic acid-amino acid-synthetic polymer, etc. In some embodiments, the organic linker has a molecular weight of from about 100 Da to about 500 Da. In some embodiments, the organic linker comprises a succinimide.

Each peptide can be linked to the biocompatible polymer by a variety of organic linkers generally known in the art for forming antibody-drug conjugates, such as those provided by Conju-Probe of San Diego, CA. Methods for forming bioconjugate bonds are described in Bioconjugate Techniques, 3^{rd} Edition, Greg T. Hermanson. The organic linkers can be reactive with amines, carbonyls, carboxyl and activated esters, can react via Click-chemistry (with or without copper), or be reactive with thiols.

Representative organic linkers include an amide or disulfide, or are formed from a reactive group such as succinic anhydride, succinimide, N-hydroxy succinimide, N-chlorosuccinimide, N-bromosuccinimide, maleic anhydride, maleimide, hydantoin, phthalimide, and others. The organic linkers useful in the present invention are small and generally have a molecular weight from about 100 Da to about 500 Da containing two functional groups consisting of a maleimide and either an amine or hydrazide. In some embodiments, the peptide is covalently linked to the polymer via a sulfide bond and an organic linker having a molecular weight of from about 100 Da to about 500 Da. In some embodiments, the organic linker has a molecular weight of from about 100 Da to about 300 Da. In some embodiments, the organic linker comprises a succinimide. In some embodiments, the organic linker is formed using N-beta-maleimidopropionic acid hydrazide (BMPH), N-epsilon-maleimidocaproic acid hydrazide (EMCH), N-aminoethylmaleimide, N-kappa-maleimidoundecanoic acid hydrazide (KUMH), hydrazide-PEG2-maleimide (MP2H), amine-PEG2-maleimide, hydrazide-PEG3-maleimide, or amine-PEG3-maleimide.

In some embodiments, the organic linker has the formula:

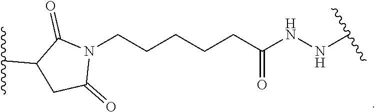 [SMILES: *NNC(=O)CCCCCN1C(=O)CC(*)C1=O]

The organic linker with the above structure is also known as N-epsilon-maleimidocaproic acid hydrazide (EMCH).

In some embodiments, the organic linker has the formula:

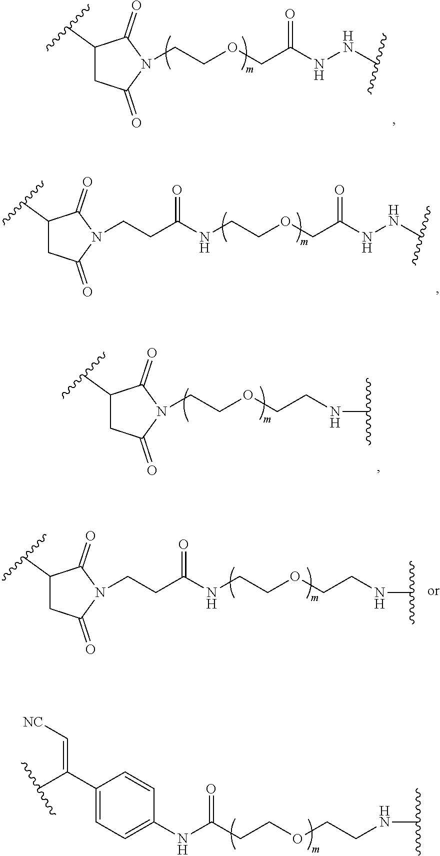

wherein subscript m is an integer of from 1 to 100. In some embodiments, the organic linker has the formula:

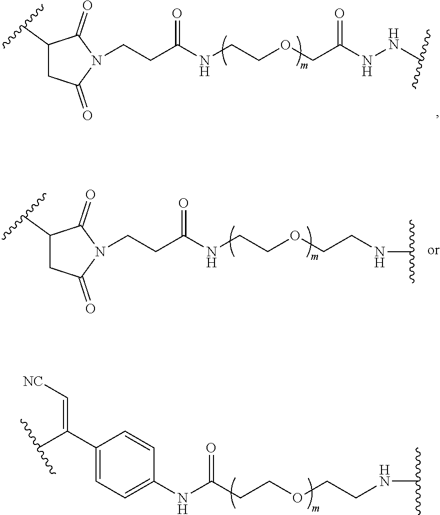

wherein subscript m is an integer of from 1 to 100. In some embodiments, subscript m is an integer of from 1 to 10. In some embodiments, subscript m is an integer of from 1 to 5. In some embodiments, subscript m is an integer of from 2 to 5. In some embodiments, subscript m is 2. In some embodiments, subscript m is 3. In some embodiments, subscript m is 4.

In some embodiments, the organic linker has the formula:

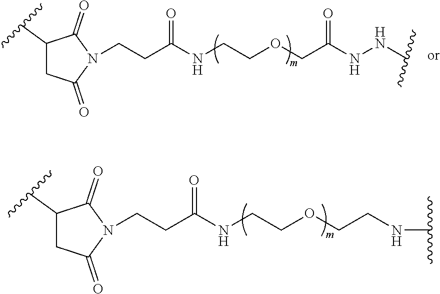

wherein subscript m is an integer of from 1 to 100. In some embodiments, subscript m is an integer of from 1 to 10. In some embodiments, subscript m is an integer of from 1 to 5. In some embodiments, subscript m is an integer of from 2 to 5. In some embodiments, subscript m is 3. In some embodiments, subscript m is 2.

In some embodiments, the organic linker has the formula:

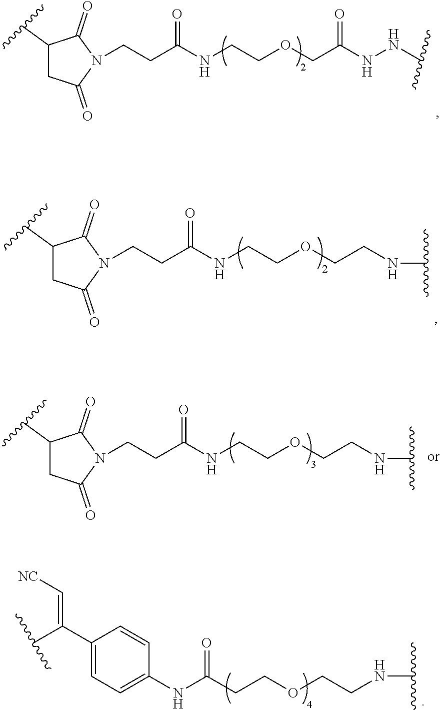

In some embodiments, the organic linker has the formula:

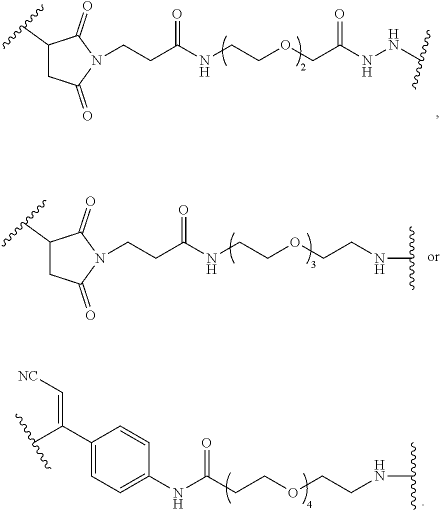

In some embodiments, the organic linker has the formula:

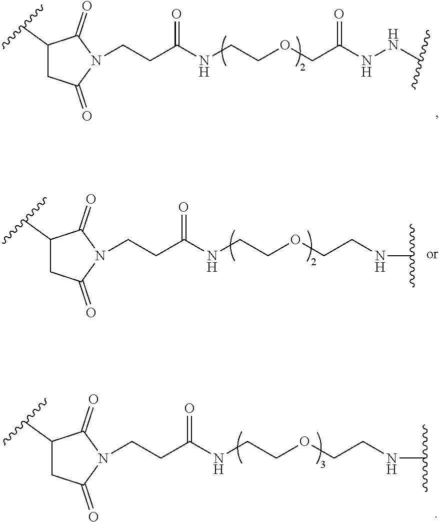

In some embodiments, the organic linker has the formula:

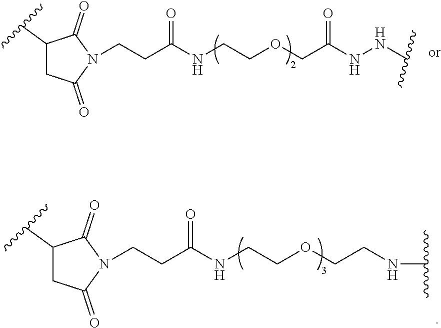

In some embodiments, the organic linker has the formula:

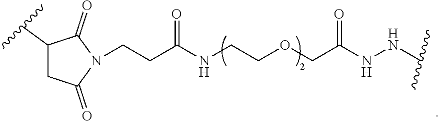

The organic linker with the above structure is also known as MP2H.

In some embodiments, preparing the conjugates of the present invention comprises covalently attaching the organic linker to the biocompatible polymer and then covalently attaching the peptide to the organic linker. In some embodiments, after preparing the conjugate of the present invention, unreacted organic linker is present on the biocompatible polymer. The structure of the unreacted organic linker depends on the organic linker and would be understood by a person skilled in the art.

Representative unreacted organic linkers include, but are not limited to,

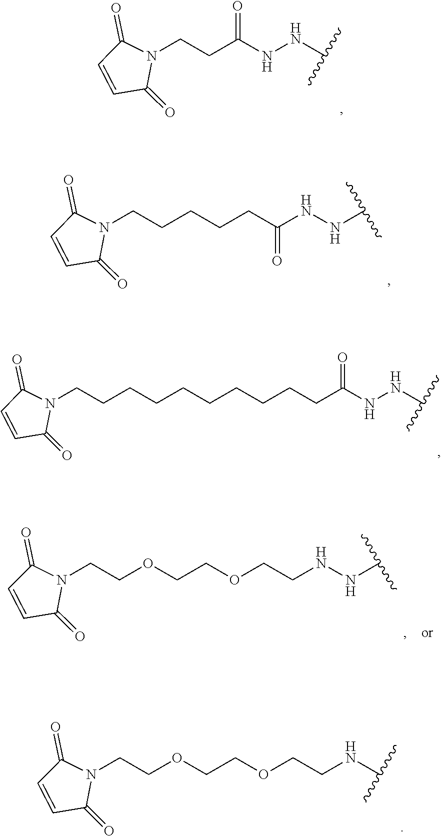 [SMILES: *NNC(=O)CCN1C(=O)C=CC1=O ; *NNC(=O)CCCCCN1C(=O)C=CC1=O ; *NNC(=O)CCCCCCCCCCN1C(=O)C=CC1=O ; *NNCCOCCOCCN1C(=O)C=CC1=O ; *NCCOCCOCCN1C(=O)C=CC1=O]

In some embodiments, the unreacted organic linker has the structure:

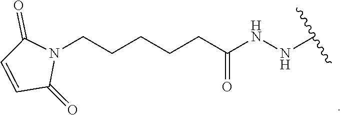 [SMILES: *NNC(=O)CCCCCN1C(=O)C=CC1=O]

In some embodiments, the unreacted organic linker has the structure:

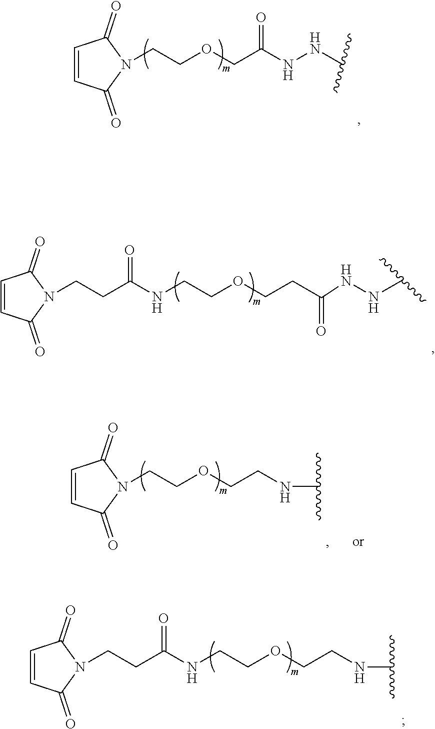

wherein subscript m is an integer of from 1 to 300. In some embodiments, subscript m is an integer from 1 to 100.

In some embodiments, the unreacted organic linker has the structure:

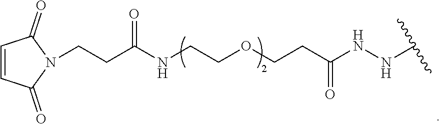

### Biocompatible Cellulose Polymers

Polymers useful in the conjugates of the present invention include any suitable biocompatible cellulose polymer. Biocompatible cellulose polymers are cellulose-based polymers that generally do not trigger an immune response. Suitable biocompatible cellulose polymers include, but are not limited to, cellulose, carboxyalkylcelluloses, such as carboxymethylcellulose and derivatives thereof, hydroxyalkylcelluloses, such as hydroxypropylcellulose and derivatives thereof, and others. The biocompatible cellulose polymers can be further modified by methods such as sulfation, sulfonation, deuteration, etc.

The biocompatible cellulose polymer can include, but is not limited to, cellulose, carboxymethylcellulose, methyl cellulose, among others. In some embodiments, the biocompatible cellulose polymer comprises carboxymethyl cellulose.

The biocompatible cellulose polymer of the present invention can be of any suitable molecular weight. For example, suitable biocompatible cellulose polymers can have a molecular weight of from about 0.01 MDa to about 3 MDa, about 0.1 MDa to about 3 MDa, or about 100 kDa to about 3,000 kDa. A polymer molecular weight can typically be expressed as the number average molecular weight (Mₙ) or the weight average molecular weight (M_{w}). The number average molecular weight is the mathematical mean of the molecular masses of the individual macromolecules. The weight average molecular weight is influenced by larger molecules and so is a larger number than the number average molecular weight. The ratio of M_{w}/Mₙ is the polydispersity of the polymer and represents the breadth of molecular weights in the polymer sample. Reference to molecular weights in the present invention are to the weight average molecular weight (M_{w}) unless stated otherwise.

Molecular weights useful for biocompatible cellulose polymer include, but are not limited to, from about 0.01 MDa to about 3 MDa, from about 0.1 MDa to about 3 MDa, from about 0.1 MDa to about 2 MDa, from about 0.2 MDa to about 1.5 MDa, from about 0.8 MDa to about 3 MDa, from about 1 MDa to about 3 MDa, from about 1.5 MDa to about 3 MDa, or from about 1 MDa to about 2 MDa. In some embodiments, the biocompatible cellulose polymer has a molecular weight of from about 0.01 MDa to about 3 MDa. In some embodiments, the biocompatible cellulose polymer has a molecular weight of from about 0.01 MDa to about 1 MDa. In some embodiments, the biocompatible cellulose polymer has a molecular weight of from about 0.1 MDa to about 3 MDa. In some embodiments, the biocompatible cellulose polymer has a molecular weight of from about 0.1 MDa to about 2 MDa. In some embodiments, the biocompatible cellulose polymer has a molecular weight of from about 0.1 MDa to about 1 MDa. In some embodiments, the biocompatible cellulose polymer has a molecular weight of from about 0.2 MDa to about 1.5 MDa. In some embodiments, the biocompatible cellulose polymer has a molecular weight of from about 0.8 MDa to about 3 MDa. The biocompatible cellulose polymer can have a molecular weight of about 0.01 MDa, or 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or about 3 MDa. In some embodiments, the biocompatible cellulose polymer has a molecular weight of about 0.045 MDa. In some embodiments, the biocompatible cellulose polymer has a molecular weight of about 0.09 MDa. In some embodiments, the biocompatible cellulose polymer has a molecular weight of about 0.25 MDa. In some embodiments, the biocompatible cellulose polymer has a molecular weight of about 0.7 MDa.

### Conjugates

The peptide-polymer conjugates of the present invention can include any suitable combination of peptide and biocompatible cellulose polymer where the molar ratio of peptide to polymer is at least 5:1. Representative molar ratios of peptide to biocompatible cellulose polymer useful in the present invention include from 5:1 to about 1000:1, from 5:1 to about 500:1, from 5:1 to about 400:1, from about 10:1 to about 500:1, from about 10:1 to about 400:1, from about 10:1 to about 300:1, from about 10:1 to about 200:1, from about 10:1 to about 100:1, from about 20:1 to about 100:1, from about 30:1 to about 100:1, from about 50:1 to about 100:1, from about 10:1 to about 50:1, from about 20:1 to about 50:1, or from about 30:1 to about 50:1. Other molar ratios of peptide to biocompatible cellulose polymer useful in the present invention include from about 50:1 to about 500:1, from about 50:1 to about 400:1, from about 50:1 to about 300:1, or from about 50:1 to about 200:1. Representative molar ratios of peptide to biocompatible cellulose polymer include about 10:1, or 15:1, 20:1, 25:1, 30:1, 35:1, 40:1, 45:1, 50:1, 60:1, 70:1, 80:1, 90:1, 100:1, 125:1, 150:1, 175:1, 200:1, 250:1, 300:1, 350:1, 450:1, 500:1, 550:1, 600:1, 650:1, 700:1, 750:1, 800:1, 850:1, 900:1, 950:1 or about 1000:1. In some embodiments, subscript n is an integer of from about 5 to about 500. In some embodiments, subscript n is an integer of from about 5 to about 200. In some embodiments, subscript n is an integer of from about 10 to about 400. In some embodiments, subscript n is an integer of from about 10 to about 120. In some embodiments, subscript n is an integer of from about 10 to about 100. In some embodiments, subscript n is an integer of from about 10 to about 50. In some embodiments, subscript n is an integer of from about 50 to about 100.

The conjugates of the peptide of biocompatible cellulose polymer of the present invention can have longer in vivo half-lives compared to the unconjugated peptide. For example, the conjugate can have an in vivo half-life of at least 2 times longer than that of the unconjugated peptide, or 3, 4, 5, 6, 7, 8, 9, 10, 15, 20, 25, 30, 35, 40, 45, 50, 60, 70, 80, 90, or at least 100 times longer than that of the unconjugated peptide. The in vivo half-life of the conjugate can be from about 2 to about 100 times longer than the unconjugated peptide, or from about 2 to about 50, from about 10 to about 100, from about 25 to about 100, from about 50 to about 100 times longer than the unconjugated peptide. In some embodiments, the in vivo half-life of the conjugate is at least 2 times longer than the unconjugated peptide. In some embodiments, the in vivo half-life of the conjugate is at least 5 times longer than the unconjugated peptide. In some embodiments, the in vivo half-life of the conjugate is from about 2 to about 100 times longer than the unconjugated peptide.

In some embodiments, the conjugate has Formula (Ia):

(X—Y)ₙ—Z (Ia),

wherein X is a peptide having an amino acid sequence of SEQ ID NO: 1; Y is an organic linker having the structure:

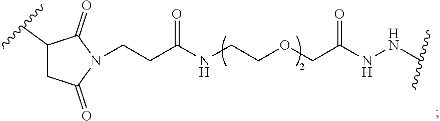

Z is a carboxymethyl cellulose polymer having a molecular weight of about 0.7 MDa; and subscript n is an integer of from about 10 to about 50.

In some embodiments, the conjugate is a conjugate of Formula IIa:

(X¹—X²—Y)ₙ—Z (IIa),

wherein
each X¹ is independently a peptide as described herein;
each X² is independently a peptide linker of from 3 to 100 amino acids in length;
each Y is independently an organic linker;
Z is a biocompatible cellulose polymer having a molecular weight of from about 0.1 MDa to about 3 MDa; and
subscript n is an integer of from 1 to 1500.

In some embodiments, the conjugate is a conjugate of Formula IIb:

(X¹—X^{2A}—Y)ₙ—Z (IIb),

wherein
each X¹ is independently a peptide having a molecular weight of from about 5 kDa to about 200 kDa;
each X^{2A} is independently a peptide linker that comprises an alpha-helix;
each Y is independently an organic linker;
Z is a biocompatible cellulose polymer having a molecular weight of from about 0.1 MDa to about 3 MDa; and
subscript n is an integer of from 1 to 1500.

In some embodiments, each X¹ is independently a peptide of the present invention. In some embodiments, each X¹ is independently a peptide having an amino acid sequence comprising any one of SEQ ID NO: 2-7 or 16.

In some embodiments, each peptide linker is independently from 7 to 100 amino acids in length. In some embodiments, each peptide linker is independently from 10 to 30 amino acids in length.

In some embodiments, each peptide linker independently has an amino acid sequence comprising:

(SEQ ID NO: 21)
AEAAAKEAAAKEAAAKAGC,
(SEQ ID NO: 22)
AEEEKRKAEEEKRKAEEEAGC,
(SEQ ID NO: 23)
AEEEKRKAEEEKRKAEEEKRKAEEEAGC,
(SEQ ID NO: 24)
AEEEEKKKKEEEEKKKKAGC,
(SEQ ID NO: 25)
AEAAAKEAAAKAGC,
(SEQ ID NO: 26)
PSRLEEELRRRLTEGC,
or
(SEQ ID NO: 27)
AEEEEKKKQQEEEAERLRRIQEEMEKERKRREEDEERRRKEEEERRMKLE
MEAKRKQEEEERKKREDDEKRKKKAGC

In some embodiments, each peptide linker has an amino acid sequence comprising AEAAAKEAAAKEAAAKAGC (SEQ ID NO: 21).

In some embodiments, the conjugate of the present invention is a conjugate that is a random polymer of Formula III:

(X—Y—Z¹)ₙ—(Z²)ₚ—(Z³)_{q} (III),
having a molecular weight of from about 0.1 MDa to about 3 MDa;
wherein
each X is independently a peptide having a molecular weight of from about 5 kDa to about 200 kDa;
each Y is an organic linker;
each X—Y—Z¹ moiety has the structure:

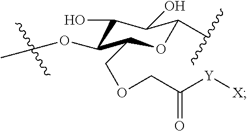

each Z² has the structure:

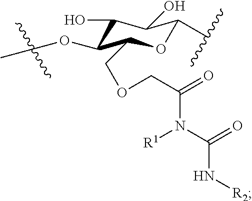

each Z³ independently has the structure:

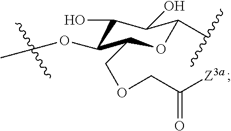

each R¹ and R² is independently C₁-C₆ alkyl, —(C₁-C₆ alkyl)-NR³R⁴, or C₅-C₈ cycloalkyl;
each R³ and R⁴ is independently H or C₁-C₆ alkyl;
each Z^{3a} is independently OH or Y′;
each Y′ is an unreacted organic linker;
subscript n is an integer of from 1 to 1500 and less than about 15% of the sum of subscripts n, p, and q;
subscript p is an integer of from 0 to 1000 and less than about 10% of the sum of subscripts n, p, and q; and
subscript q is an integer of from 100 to 10000.

In some embodiments, each X is independently a peptide having an amino acid sequence comprising any one of SEQ ID NO: 1 and 8-15.

In some embodiments, the conjugate has the structure of Formula IIIa:

(X¹—X²—Y—Z¹)ₙ—(Z²)ₚ—(Z³)_{q} (IIIa),
wherein
each X¹ is independently a peptide having a molecular weight of from about 5 kDa to about 200 kDa; and
each X² is a peptide linker that comprises an alpha-helix.

In some embodiments, each X² is independently a peptide having an amino acid sequence comprising any one of SEQ ID NO: 21-27.

In some embodiments, the random polymer of Formula III has a molecular weight of from about 0.4 MDa to about 2 MDa. In some embodiments, the random polymer of Formula III has a molecular weight of from about 0.7 MDa to about 1.5 MDa. In some embodiments, the random polymer of Formula III has a molecular weight of about 0.8 MDa.

In some embodiments, each R¹ and R² is independently C₁-C₃ alkyl or —(C₁-C₃ alkyl)-NR³R⁴. In some embodiments, each R¹ and R² is ethyl or —(CH₂)₃—NMe₂. In some embodiments, each R¹ is ethyl; and each R² is —(CH₂)₃—NMe₂. In some embodiments, each R¹ is —(CH₂)₃—NMe₂; and each R² is ethyl.

In some embodiments, each R³ and R⁴ is independently C₁-C₃ alkyl. In some embodiments, each R³ and R⁴ is methyl.

In some embodiments, subscript n is an integer of from 1 to 1500 and less than about 15% of the sum of subscripts n, p, and q; subscript p is an integer of from 1 to 1000 and less than about 10% of the sum of subscripts n, p, and q; and subscript q is an integer of from 100 to 10000. In some embodiments, subscript n is an integer of from 1 to 1000 and less than about 10% of the sum of subscripts n, p, and q; subscript p is an integer of from 1 to 800 and less than about 8% of the sum of subscripts n, p, and q; and subscript q is an integer of from 100 to 10000. In some embodiments, subscript n is an integer of from 10 to 450 and less than about 15% of the sum of subscripts n, p, and q; subscript p is an integer of from 1 to 300 and less than about 10% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 240 and less than about 8% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 60 and less than about 2% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 30 and less than about 1% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 15 and less than about 0.5% of the sum of subscripts n, p, and q; and subscript q is an integer of from 1000 to 3000.

The conjugates described herein may be prepared and/or formulated as pharmaceutically acceptable salts or when appropriate as a free base. Pharmaceutically acceptable salts are non-toxic salts of a free base form of a compound that possess the desired pharmacological activity of the free base. These salts may be derived from inorganic or organic acids or bases. For example, a conjugate that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21^{st} Edition, Lippincott Williams and Wilkins, Philadelphia, Pa., 2006.

Examples of “pharmaceutically acceptable salts” of the conjugates disclosed herein also include salts derived from an appropriate base, such as an alkali metal (for example, sodium, potassium), an alkaline earth metal (for example, magnesium), ammonium and NR₄+(wherein R is C₁-C₄ alkyl). Also included are base addition salts, such as sodium or potassium salts.

In some embodiments, a conjugate of the present invention exhibits a half-life in vivo of from about 12 hours to about 24 hours, from about 1 day to about 3 days, from about 3 days to about 7 days, from one week to about 2 weeks, from about 2 weeks to about 4 weeks, or from about 1 month to about 6 months.

In some embodiments, a conjugate of the present invention exhibits a therapeutically efficacious residence time in vivo of from about 12 hours to about 24 hours, from about 1 day to about 3 days, from about 3 days to about 7 days, from one week to about 2 weeks, from about 2 weeks to about 4 weeks, from about 1 month to about 3 months, or from about 3 months to about 6 months.

The biological activity of a conjugate is enhanced relative to the activity of the corresponding peptide in soluble form, e.g., compared to the activity of the peptide not conjugated to the polymer. In some embodiments, the biological activity of the conjugate is at least about 25%, at least about 50%, at least about 75%, at least about 2-fold, at least about 5-fold, at least about 10-fold, at least about 15-fold, at least about 20-fold, at least about 25-fold, at least about 30-fold, at least about 40-fold, at least about 50-fold, at least about 75-fold, at least about 100-fold, at least about 200-fold, at least about 500-fold, or at least about 1000-fold, or more than 1000-fold, greater than the biological activity of the peptide in soluble (unconjugated) form.

### III. Pharmaceutical Compositions

In some embodiments, a pharmaceutical composition of the present invention includes a conjugate of the present invention and a pharmaceutically acceptable excipient.

### A. Formulation

For preparing pharmaceutical compositions from the conjugates of the present invention, pharmaceutically acceptable carriers can be either solid or liquid. Solid form preparations include powders, cachets, and dispersible granules. A solid carrier can be one or more substances, which may also act as diluents, binders, preservatives, disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

Liquid form preparations include solutions, suspensions, and emulsions, for example, water or water/propylene glycol solutions. For intra-tumoral injection, liquid preparations can be formulated in solution in aqueous polyethylene glycol solution.

Also included are solid form preparations, which are intended to be converted, shortly before use, to liquid form preparations for intra-tumoral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the conjugates of the present invention.

Oil suspensions can be formulated by suspending the conjugates of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In some embodiments, the compositions of the present invention can be formulated for intra-tumoral administration. The formulations for administration will commonly comprise a solution of the compositions of the present invention dissolved in a pharmaceutically acceptable catrer. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. The formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46: 1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present invention include any natural or synthetic lipids including, but not limited to, sesame seed oil, olive oil, castor oil, peanut oil, fatty acid esters, glycerol esters, Labrafil®, Labrasol@, Cremophor®, Solutol@, Tween®, Capryol@, Capmul@, Captex@, and Peceol@.

### B. Administration

The conjugates and compositions of the present invention can be delivered by any suitable means. In some embodiments, the conjugate is administered intratumorally. In some embodiments, the conjugate is administered intravenously.

The pharmaceutical preparation is preferably in unit dosage form. In such form the preparation is subdivided into unit doses containing appropriate quantities of the conjugates and compositions of the present invention. The unit dosage form can be a packaged preparation, the package containing discrete quantities of preparation, such as packeted tablets, capsules, and powders in vials or ampoules.

The conjugates and compositions of the present invention can be co-administered with other agents. Co-administration includes administering the conjugate or composition of the present invention within 0.5, 1, 2, 4, 6, 8, 10, 12, 16, 20, or 24 hours of the other agent. Co-administration also includes administering simultaneously, approximately simultaneously (e.g., within about 1, 5, 10, 15, 20, or 30 minutes of each other), or sequentially in any order. Moreover, the conjugates and compositions of the present invention can each be administered once a day, or two, three, or more times per day so as to provide the preferred dosage level per day.

In some embodiments, co-administration can be accomplished by co-formulation, i.e., preparing a single pharmaceutical composition including the conjugates and compositions of the present invention and any other agent. Alternatively, the various components can be formulated separately.

The conjugates and compositions of the present invention, and any other agents, can be present in any suitable amount, and can depend on various factors including, but not limited to, weight and age of the subject, state of the disease, etc. Suitable dosage ranges include from about 0.1 mg to about 10,000 mg, or about 1 mg to about 1000 mg, or about 10 mg to about 750 mg, or about 25 mg to about 500 mg, or about 50 mg to about 250 mg. Suitable dosages also include about 1 mg, 5, 10, 20, 30, 40, 50, 60, 70, 80, 90, 100, 200, 300, 400, 500, 600, 700, 800, 900 or 1000 mg. The composition can also contain other compatible therapeutic agents. The conjugates described herein can be used in combination with one another, with other active agents known to be useful in modulating a glucocorticoid receptor, or with adjunctive agents that may not be effective alone, but may contribute to the efficacy of the active agent.

### IV. Methods of Treatment

In some embodiments, a method and/or use for treating a cancer in a subject comprises administering to the subject a therapeutically effective amount of a conjugate of the present invention, or a pharmaceutical composition thereof.

In some embodiments, the cancer is a solid tumor, a hematological cancer, or a metastatic lesion. In some embodiments, the solid tumor is a sarcoma, a fibroblastic sarcoma, a carcinoma, or an adenocarcinoma. In some embodiments, the hematological cancer is a leukemia, a lymphoma, or a myeloma. In some embodiments, the metastatic lesion is a leukemia, a lymphoma, or a myeloma.

In some embodiments, the cancer is a solid tumor.

In some embodiments, the cancer is a lung cancer, a melanoma, a renal cancer, a liver cancer, a myeloma, a prostate cancer, a breast cancer, an ovarian cancer, a colorectal cancer, a pancreatic cancer, a head and neck cancer, an anal cancer, a gastro-esophageal cancer, a mesothelioma, a nasopharyngeal cancer, a thyroid cancer, a cervical cancer, an epithelial cancer, a peritoneal cancer, a B cell lymphoma, a diffuse large B-cell lymphoma (DLBCL), an activated B-cell like (ABC) diffuse large B cell lymphoma, a germinal center B cell (GCB) diffuse large B cell lymphoma, a mantle cell lymphoma, a Hodgkin lymphoma, a non-Hodgkin lymphoma, a relapsed non-Hodgkin lymphoma, a refractory non-Hodgkin lymphoma, a recurrent follicular non-Hodgkin lymphoma, a Burkitt lymphoma, a small lymphocytic lymphoma, a follicular lymphoma, a lymphoplasmacytic lymphoma, or an extranodal marginal zone lymphoma.

In some embodiments, the cancer is a bladder cancer, bone cancer, brain cancer, breast cancer, cervical cancer, central nervous system (CNS) cancer, colorectal cancer, endometrial cancer, esophageal cancer, gastric cancer, head and neck cancer, hepatocellular cancer, lung cancer, lymphoma, melanoma, neuroendocrine cancer, ovarian cancer, pancreatic cancer, prostate cancer, renal cancer, sarcoma, or thyroid cancer.

In some embodiments, the cancer is an epithelial tumor (e.g., a carcinoma, a squamous cell carcinoma, a basal cell carcinoma, a squamous intraepithelial neoplasia), a glandular tumor (e.g., an adenocarcinoma, an adenoma, an adenomyoma), a mesenchymal or soft tissue tumor (e.g., a sarcoma, a rhabdomyosarcoma, a leiomyosarcoma, a liposarcoma, a fibrosarcoma, a dermatofibrosarcoma, a neurofibrosarcoma, a fibrous histiocytoma, an angiosarcoma, an angiomyxoma, a leiomyoma, a chondroma, a chondrosarcoma, an alveolar soft-part sarcoma, an epithelioid hemangioendothelioma, a Spitz tumor, a synovial sarcoma), or a lymphoma.

In some embodiments, the cancer is a solid tumor in or arising from a tissue or organ selected from the group consisting of: bone (e.g., adamantinoma, aneurysmal bone cysts, angiosarcoma, chondroblastoma, chondroma, chondromyxoid fibroma, chondrosarcoma, chordoma, dedifferentiated chondrosarcoma, enchondroma, epithelioid hemangioendothelioma, fibrous dysplasia of the bone, giant cell tumour of bone, haemangiomas and related lesions, osteoblastoma, osteochondroma, osteosarcoma, osteoid osteoma, osteoma, periosteal chondroma, Desmoid tumor, Ewing sarcoma); lips and oral cavity (e.g., odontogenic ameloblastoma, oral leukoplakia, oral squamous cell carcinoma, primary oral mucosal melanoma); salivary glands (e.g., pleomorphic salivary gland adenoma, salivary gland adenoid cystic carcinoma, salivary gland mucoepidermoid carcinoma, salivary gland Warthin's tumors); esophagus (e.g., Barrett's esophagus, dysplasia and adenocarcinoma); gastrointestinal tract, including stomach (e.g., gastric adenocarcinoma, primary gastric lymphoma, gastrointestinal stromal tumors (GISTs), metastatic deposits, gastric carcinoids, gastric sarcomas, neuroendocrine carcinoma, gastric primary squamous cell carcinoma, gastric adenoacanthomas), intestines and smooth muscle (e.g., intravenous leiomyomatosis), colon (e.g., colorectal adenocarcinoma), rectum, anus; pancreas (e.g., serous neoplasms, including microcystic or macrocystic serous cystadenoma, solid serous cystadenoma, Von Hippel-Landau (VHL)-associated serous cystic neoplasm, serous cystadenocarcinoma, mucinous cystic neoplasms (MCN), intraductal papillary mucinous neoplasms (IPMN), intraductal oncocytic papillary neoplasms (IOPN), intraductal tubular neoplasms, cystic acinar neoplasms, including acinar cell cystadenoma, acinar cell cystadenocarcinoma, pancreatic adenocarcinoma, invasive pancreatic ductal adenocarcinomas, including tubular adenocarcinoma, adenosquamous carcinoma, colloid carcinoma, medullary carcinoma, hepatoid carcinoma, signet ring cell carcinoma, undifferentiated carcinoma, undifferentiated carcinoma with osteoclast-like giant cells, acinar cell carcinoma, neuroendocrine neoplasms, neuroendocrine microadenoma, neuroendocrine tumors (NET), neuroendocrine carcinoma (NEC), including small cell or large cell NEC, insulinoma, gastrinoma, glucagonoma, serotonin-producing NET, somatostatinoma, VIPoma, solid-pseudopapillary neoplasms (SPN), pancreatoblastoma); gall bladder (e.g. carcinoma of the gallbladder and extrahepatic bile ducts, intrahepatic cholangiocarcinoma); neuro-endocrine (e.g., adrenal cortical carcinoma, carcinoid tumors, phaeochromocytoma, pituitary adenomas); thyroid (e.g., anaplastic (undifferentiated) carcinoma, medullary carcinoma, oncocytic tumors, papillary carcinoma, adenocarcinoma); liver (e.g., adenoma, combined hepatocellular and cholangiocarcinoma, fibrolamellar carcinoma, hepatoblastoma, hepatocellular carcinoma, mesenchymal, nested stromal epithelial tumor, undifferentiated carcinoma, hepatocellular carcinoma, intrahepatic cholangiocarcinoma, bile duct cystadenocarcinoma, epithelioid hemangioendothelioma, angiosarcoma, embryonal sarcoma, rhabdomyosarcoma, solitary fibrous tumor, teratoma, York sac tumor, carcinosarcoma, rhabdoid tumor); kidney (e.g., ALK-rearranged renal cell carcinoma, chromophobe renal cell carcinoma, clear cell renal cell carcinoma, clear cell sarcoma, metanephric adenoma, metanephric adenofibroma, mucinous tubular and spindle cell carcinoma, nephroma, nephroblastoma (Wilms tumor), papillary adenoma, papillary renal cell carcinoma, renal oncocytoma, renal cell carcinoma, succinate dehydrogenase-deficient renal cell carcinoma, collecting duct carcinoma); breast (e.g., invasive ductal carcinoma, including without limitation, acinic cell carcinoma, adenoid cystic carcinoma, apocrine carcinoma, cribriform carcinoma, glycogen-rich/clear cell, inflammatory carcinoma, lipid-rich carcinoma, medullary carcinoma, metaplastic carcinoma, micropapillary carcinoma, mucinous carcinoma, neuroendocrine carcinoma, oncocytic carcinoma, papillary carcinoma, sebaceous carcinoma, secretory breast carcinoma, tubular carcinoma, lobular carcinoma, including without limitation, pleomorphic carcinoma, signet ring cell carcinoma, peritoneum (e.g., mesothelioma, primary peritoneal cancer)); female sex organ tissues, including ovary (e.g., choriocarcinoma, epithelial tumors, germ cell tumors, sex cord-stromal tumors), Fallopian tubes (e.g., serous adenocarcinoma, mucinous adenocarcinoma, endometrioid adenocarcinoma, clear cell adenocarcinoma, transitional cell carcinoma, squamous cell carcinoma, undifferentiated carcinoma, mullerian tumors, adenosarcoma, leiomyosarcoma, teratoma, germ cell tumors, choriocarcinoma, trophoblastic tumors), uterus (e.g., carcinoma of the cervix, endometrial polyps, endometrial hyperplasia, intraepithelial carcinoma (EIC), endometrial carcinoma (e.g., endometrioid carcinoma, serous carcinoma, clear cell carcinoma, mucinous carcinoma, squamous cell carcinoma, transitional carcinoma, small cell carcinoma, undifferentiated carcinoma, mesenchymal neoplasia), leiomyoma (e.g., endometrial stromal nodule, leiomyosarcoma, endometrial stromal sarcoma (ESS), mesenchymal tumors), mixed epithelial and mesenchymal tumors (e.g., adenofibroma, carcinofibroma, adenosarcoma, carcinosarcoma (malignant mixed mesodermal sarcoma-MMMT)), endometrial stromal tumors, endometrial malignant mullerian mixed tumours, gestational trophoblastic tumors (partial hydatiform mole, complete hydatiform mole, invasive hydatiform mole, placental site tumour)), vulva, vagina; male sex organ tissues, including prostate, testis (e.g., germ cell tumors, spermatocytic seminoma), penis; bladder (e.g., squamous cell carcinoma, urothelial carcinoma, bladder urothelial carcinoma); brain, (e.g., gliomas (e.g., astrocytomas, including non-infiltrating, low-grade, anaplastic, glioblastomas; oligodendrogliomas, ependymomas), meningiomas, gangliogliomas, schwannomas (neurilemmomas), craniopharyngiomas, chordomas, Non-Hodgkin lymphomas, pituitary tumors; eye (e.g., retinoma, retinoblastoma, ocular melanoma, posterior uveal melanoma, iris hamartoma); head and neck (e.g., nasopharyngeal carcinoma, Endolymphatic Sac Tumor (ELST), epidermoid carcinoma, laryngeal cancers including squamous cell carcinoma (SCC) (e.g., glottic carcinoma, supraglottic carcinoma, subglottic carcinoma, transglottic carcinoma), carcinoma in situ, verrucous, spindle cell and basaloid SCC, undifferentiated carcinoma, laryngeal adenocarcinoma, adenoid cystic carcinoma, neuroendocrine carcinomas, laryngeal sarcoma), head and neck paragangliomas (e.g., carotid body, jugulotympanic, vagal); thymus (e.g., thymoma); heart (e.g., cardiac myxoma); lung (e.g., small cell carcinoma (SCLC), non-small cell lung carcinoma (NSCLC), including squamous cell carcinoma (SCC), adenocarcinoma and large cell carcinoma, carcinoids (typical or atypical), carcinosarcomas, pulmonary blastomas, giant cell carcinomas, spindle cell carcinomas, pleuropulmonary blastoma); lymph (e.g., lymphomas, including Hodgkin's lymphoma, non-Hodgkin's lymphoma, Epstein-Barr virus (EBV)-associated lymphoproliferative diseases, including B cell lymphomas and T cell lymphomas (e.g., Burkitt lymphoma, large B cell lymphoma, diffuse large B-cell lymphoma (DLBCL), mantle cell lymphoma, indolent B-cell lymphoma, low grade B cell lymphoma, fibrin-associated diffuse large cell lymphoma; primary effusion lymphoma; plasmablastic lymphoma; extranodal NK/T cell lymphoma, nasal type; peripheral T cell lymphoma, cutaneous T cell lymphoma, angioimmunoblastic T cell lymphoma; follicular T cell lymphoma; systemic T cell lymphoma), lymphangioleiomyomatosis); central nervous system (CNS) (e.g., gliomas including astrocytic tumors (e.g., pilocytic astrocytoma, pilomyxoid astrocytoma, subependymal giant cell astrocytoma, pleomorphic xanthoastrocytoma, diffuse astrocytoma, fibrillary astrocytoma, gemistocytic astrocytoma, protoplasmic astrocytoma, anaplastic astrocytoma, glioblastoma (e.g., giant cell glioblastoma, gliosarcoma, glioblastoma multiforme) and gliomatosis cerebri), oligodendroglial tumors (e.g., oligodendroglioma, anaplastic oligodendroglioma), oligoastrocytic tumors (e.g., oligoastrocytoma, anaplastic oligoastrocytoma), ependymal tumors (e.g., subependymom, myxopapillary ependymoma, ependymomas (e.g., cellular, papillary, clear cell, tanycytic), anaplastic ependymoma), optic nerve glioma, and non-gliomas (e.g., choroid plexus tumors, neuronal and mixed neuronal-glial tumors, pineal region tumors, embryonal tumors, medulloblastoma, meningeal tumors, primary CNS lymphomas, germ cell tumors, pituitary adenomas, cranial and paraspinal nerve tumors, stellar region tumors), neurofibroma, meningioma, peripheral nerve sheath tumors, peripheral neuroblastic tumours (including without limitation neuroblastoma, ganglioneuroblastoma, ganglioneuroma), trisomy 19 ependymoma); neuroendocrine tissues (e.g., paraganglionic system including adrenal medulla (pheochromocytomas) and extra-adrenal paraganglia ((extra-adrenal) paragangliomas); skin (e.g., clear cell hidradenoma, cutaneous benign fibrous histiocytomas, cylindroma, hidradenoma, melanoma (including cutaneous melanoma, mucosal melanoma), pilomatricoma, Spitz tumors); and soft tissues (e.g., aggressive angiomyxoma, alveolar rhabdomyosarcoma, alveolar soft part sarcoma, angiofibroma, angiomatoid fibrous histiocytoma, synovial sarcoma, biphasic synovial sarcoma, clear cell sarcoma, dermatofibrosarcoma protuberans, desmoid-type fibromatosis, small round cell tumor, desmoplastic small round cell tumor, elastofibroma, embryonal rhabdomyosarcoma, Ewing's tumors/primitive neurectodermal tumors (PNET), extraskeletal myxoid chondrosarcoma, extraskeletal osteosarcoma, paraspinal sarcoma, inflammatory myofibroblastic tumor, lipoblastoma, lipoma, chondroid lipoma, liposarcoma/malignant lipomatous tumors, liposarcoma, myxoid liposarcoma, fibromyxoid sarcoma, lymphangioleiomyoma, malignant myoepithelioma, malignant melanoma of soft parts, myoepithelial carcinoma, myoepithelioma, myxoinflammatory fibroblastic sarcoma, undifferentiated sarcoma, pericytoma, rhabdomyosarcoma, non rhabdomyosarcoma, soft tissue sarcoma (NRSTS), soft tissue leiomyosarcoma, undifferentiated sarcoma, well-differentiated liposarcoma.

In some embodiments, the cancer is a melanoma, a gastric cancer, a triple-negative breast cancer (TNBC), a non-small cell lung cancer (NSCLC), a rectal adenocarcinoma, a colorectal cancer, a renal cell carcinoma, an ovarian cancer, a prostate cancer, an oral squamous cell carcinoma (SCC), a head and neck squamous cell carcinoma (HNSCC), a urothelial bladder cancer, a glioblastoma (GBM), a meningioma, adrenal cancer, or an endometrial cancer.

In some embodiments, the cancer is a melanoma. In some embodiments, the cancer is a head and neck squamous cell carcinoma. In some embodiments, the cancer is a triple negative breast cancer.

In some embodiments, a use of the present invention is a use of a therapeutically effective amount of a conjugate of the present invention, or a pharmaceutical composition thereof, in the preparation of a medicament for treating a cancer in a subject.

In some embodiments, a conjugate for use of the present invention is a therapeutically effective amount of a conjugate of the present invention, or a pharmaceutical composition thereof, for use in treating a cancer in a subject.

In some embodiments, a composition for use of the present invention is a pharmaceutical composition comprising a therapeutically effective amount of a conjugate of the present invention for use in treating a cancer in a subject.

### V. Examples

Certain abbreviations and acronyms are used in describing the experimental details. Although most of these would be understood by one skilled in the art, the Table below contains a list of many of these abbreviations and acronyms.

TABLE 1
List of abbreviations and acronyms

| Abbreviation | Meaning |
|---|---|
| BCS | body condition score |
| CMC | carboxymethyl cellulose |
| Da | daltons |
| DLS | dynamic light scattering |
| DMSO | dimethyl sulfoxide |
| DPBS | Dulbecco's phosphate buffered saline |
| DTT | dithiothreitol |
| EDTA | ethylenediaminetetraacetic acid |
| ELISA | enzyme-linked immunosorbent assay |
| FPLC | fast protein liquid chromatography |
| HyA | hyaluronic acid |
| IL | interleukin |
| IT | intra-tumoral |
| K_{d} | dissociation constant |
| kDa | kilodaltons |
| MDa | megadaltons |
| MVP | multivalent protein |
| MW | molecular weight |
| MWCO | molecular weight cutoff |
| NHS | N-hydroxysuccinimide |
| NK cell | natural killer cell |
| PBMC | peripheral blood mononuclear cell |
| PBS | phosphate buffered saline |
| ROI | region of interest |
| RPM | revolutions per minute |
| RT | room temperature |
| SDS-PAGE | sodium dodecyl sulfate-polyacrylamide gel electrophoresis |
| TCEP | tris(2-carboxyethyl)phosphine |
| TIL | tumor infiltrating lymphocyte |
| TNBC | triple negative breast cancer |

### Example 1. Preparation of Conjugates

### Method A

To obtain diacylhydrazine-linked heterobifunctional crosslinker modification of carboxymethylcellulose (CMC), hyaluronic acid (HyA), or other acid containing polysaccharide, aiming for a thiol reactive valency of ˜20-200 (depending on crosslinker), the biopolymer was suspended in 0.1 M 2-(N-morpholino)ethanesulfonic acid buffer pH 5.7 at 4 mg/mL by gentle rotation or mixing with nutation overnight. To 3 mg (3.6 nmol) of biopolymer in solution, 9,500 equivalents of 1-ethyl-3-(3-dimethylaminopropyl)carbodiimide hydrochloride (EDC) was added as a 100-1000 mg/mL stock in 0.1 M MES buffer pH 6.5. Next, 50 equivalents of hydroxybenzotriazole (HOBt) hydrate was added as a 5-50 mg/mL stock solution in DMSO, as well as 500 equivalents of hydrazide-X-maleimide reactive linker in DMSO (10-50 mg/mL stock). The final volume was diluted to 1 mL with buffer. The solution was mixed with gentle pipetting between each reagent addition. The mixture was then allowed to react at room temperature for 2 h with nutating mixer. After 2 h, the thiol reactive biopolymer was purified using 7 kDa MWCO 5 mL Zeba desalting spin column equilibrated with 10% v/v glycerol pH 6.5 DPBS or using 1:200 dialysis conditions using the same buffer. The product was eluted into clean conical tube using centrifuge at RT, elution time ˜25-45 minutes, and used immediately for reaction with thiol or aliquoted, then flash frozen on dry ice. For dialysis, the crude reaction was loaded into 100 kDa MWCO dialysis cassette (1 mL) and dialyzed 1:200 against purification buffer for at least 4 h, swapping buffer two times.

### Method B

For some cases, 725 kDa carboxymethylcellulose (CMC) with a degree of substitution (DS)=0.7 was modified with diacylhydrazine-linked heterobifunctional crosslinkers using an alternate reaction method. The CMC biopolymer was dissolved in water at 4.8 mg/mL for at least 48 h at RT with nutation. Once in solution, the CMC stock for reaction was made by adding ˜1 M pH 5.7 2-(N-morpholino)-ethanesulfonic acid (MES) and water for a final concentration of 0.1 M MES buffer pH 5.7 at 4 mg/mL CMC. To 3 mg (3.6 nmol) of biopolymer in solution, 1000 equivalents of hydroxybenzotriazole (HOBt) hydrate as a 50 mg/mL stock solution in DMSO, and 1000 equivalents of hydrazide-PEG2-maleimide reactive linker in DMSO (50 mg/mL stock) was added followed by 500 equivalents of 1-ethyl-3-(3-dimethylaminopropyl)carbodiimide hydrochloride (EDC) as a 100 mg/mL stock in 0.1 M MES buffer pH 5.7. The reaction was brought to a final volume to 1 mL with buffer and run at room temperature for 50-70 min on nutating mixer. After this, the crude thiol reactive biopolymer was purified using 7 kDa MWCO 5 mL Zeba desalting spin column equilibrated with 10% v/v glycerol pH 6.5 DPBS. After eluting product into clean conical tube by centrifuging at RT (elution time ˜25-45 minutes), the product was either used immediately for reaction with a thiol-presenting peptide or aliquoted and flash frozen on dry ice.

To obtain conjugates with peptides conjugated over a range of valencies, a fixed concentration of peptide was combined with the maleimide-functionalized polymer at various defined feed ratios in PBS and allowed to react at either 4° C. or ambient temperature for at least 4 hours up to overnight, with rotation or nutating mixing (most reactions are ran at RT to improve solubility). In some cases, before the conjugation reaction, 10-100 equivalents of a reducing agent such as DTT or TCEP HCl was added per protein equivalent to reduce any disulfide bringing between peptides. This was removed from the protein solution prior to conjugation by a desalting column or buffer exchange or can be added to the conjugation reaction directly in the form of TCEP immobilized on polymeric beads. During the conjugation reaction, one or more of the following was added to improve the reaction efficiency: 0.5-10 mM EDTA to minimize free thiol oxidation, tween20, carbohydrate, or glycerol to stabilize protein and/or help reduce non-specific interactions between protein and activated biopolymer, increased or decreased salt concentration to stabilize protein and/or help reduce non-specific interactions between protein and activated biopolymer. Unreacted peptide was removed from the peptide-polymer conjugates by one or more of the following methods: (i) dialysis with 50-100 kDa MWCO against an appropriate buffer (pH should be >1 unit above or below the pI of peptide) for three to five times for 4 hours each and once for at least 4 hours at a time range of 4 hours to overnight at 4° C.-room temperature, (ii) tangential flow filtration against buffers such as DPBS pH 6-8, 50 mM tris 150 mM NaCl pH 5-5.5, or 50 mM Succinate, 60 mM NaCl pH 5.5-6.5 with additives such as EDTA and tween or other additives like trehalose, depending on peptide, (iii) FPLC polishing using a size exclusion column, (iv) FPLC polishing with an affinity chromatography column that was designed to bind the polymer component of the conjugate, or (v) selective precipitation of the conjugates. If reaction efficiency was high enough (<4% unreacted protein present), purification was not necessary.

Alternatively, to each solution of activated polymer, the peptide was added at a suitable peptide:polymer molar feed ratio and Tween-20 to a final concentration of 0.01% (optional). The solution was allowed to react for 2 hours to overnight while agitating by rotation (˜5 RPM) or nutation at ambient temperatures. Unreacted peptide was removed from the peptide-polymer conjugates by one or more of the following methods: (i) dialysis with 50-100 kDa MWCO against an appropriate buffer (pH should be >1 unit above or below the p of peptide) for three to five times for 4 hours each and once for at least 4 hours at a time range of 4 hours to overnight at 4° C.-room temperature, (11) tangential flow filtration against DPBS pH 6-8, or 50 mMv tris 150 mM NaCl pH 8-8.5 with additives such as EDTA and tween or other additives like trehalose, depending on peptide, (111) FPLC polishing using a size exclusion column, (iv) FPLC polishing with an affinity chromatography column that was designed to bind the polymer component of the conjugate, or (v) selective precipitation of the conjugates. If reaction efficiency was high enough (<400 unreacted protein present), purification was not necessary.

| | | | Hydrazide-X | | |
|---|---|---|---|---|---|
| | Bio- | EDC | maleimide | HOBt | |
| Method | polymer | equivalents | equivalents | equivalents | Peptide |
| A | CMC | 9500 | 500 | 50 | SEQ ID NO: |
| | | | | | 1, 13, 14 |
| A | HyA | 9500 | 500 | 50 | SEQ ID NO: |
| | | | | | 1 |
| B | CMC | 500 | 1000 | 1000 | SEQ ID NO: |
| | | | | | 1, 6, 8, 9, |
| | | | | | 10, 11, 15 |

TABLE 2
Reaction conditions for peptide-polymer conjugates

| | | Reactive | | | |
|---|---|---|---|---|---|
| Conjugate | | Thiol | | Polymer | Peptide |
| ID | Method | (μM) | Peptide | (μM) | (μM) |
| 101 | A | n.d. | Anti-VEGF DARPin | 2.08 | 85.4 |
| | | | (SEQ ID NO: 1) | | |
| 103 | B | 471.8 | Anti-PD-1 VHH | 3.92 | 519.0 |
| | | | (SEQ ID NO: 9) | | |
| 104 | B | 145.1 | IL-2 | 1.21 | 159.6 |
| | | | (SEQ ID NO: 10) | | |
| 105 | B | 173.0 | IL-15 | 1.44 | 190.3 |
| | | | (SEQ ID NO: 11) | | |
| 106 | B | 78.6 | IL-15Ra-IL-15 fusion | 0.65 | 117.9 |
| | | | (SEQ ID NO: 12) | | |
| 107 | A | n.d. | Anti- TNFα Affibody | 1.11 | 64.4 |
| | | | (SEQ ID NO: 13) | | |
| 108 | A | n.d. | Anti-IL-1β scFv | 0.09 | 5.4 |
| | | | (SEQ ID NO: 14) | | |
| 109 | A | n.d. | Anti-VEGF DARPin | 1.05 | 61.2 |
| | | | (SEQ ID NO: 1) | | |
| 110 | A | n.d. | Anti-VEGF DARPin | 1.40 | 97.2 |
| | | | (SEQ ID NO: 1) | | |
| 111 | B | 121.0 | Anti-VEGF DARPin | 2.35 | 242.2 |
| | | | (SEQ ID NO: 1) | | |
| 112 | B | 186.7 | Anti-MCSP VHH | 1.55 | 205.2 |
| | | | (SEQ ID NO: 15) | | |
| 113 | B | 281.7 | Anti-VEGF VHH | 44.97 | 309.9 |
| | | | (SEQ ID NO: 8) | | |
| 114 | B | 288.1 | Anti-VEGF VHH | 24.04 | 316.9 |
| | | | (SEQ ID NO: 8) | | |
| 115 | B | 260.8 | Anti-VEGF VHH | 9.44 | 286.9 |
| | | | (SEQ ID NO: 8) | | |

n.d. = not determined

After purification, the products of the conjugation reactions were analyzed by SDS-PAGE separation. The reaction products were further analyzed for protein concentration, percent unconjugated peptide, conjugated peptide, valency (molar ratio of conjugated peptide to polymer), hydrodynamic radius (Rₕ), and binding (K_{D}), which are shown in Table 5. Protein concentration was determined based on absorbance at 280 nm, percent unconjugated protein was determined by densiometric analysis of the SDS-PAGE gels, and hydrodynamic radius was measured using dynamic light scattering (DLS) with either a Wyatt Nanostar or Plate reader III instrument at 25° C. Extent of conjugation was confirmed either by SDS-PAGE or DLS.

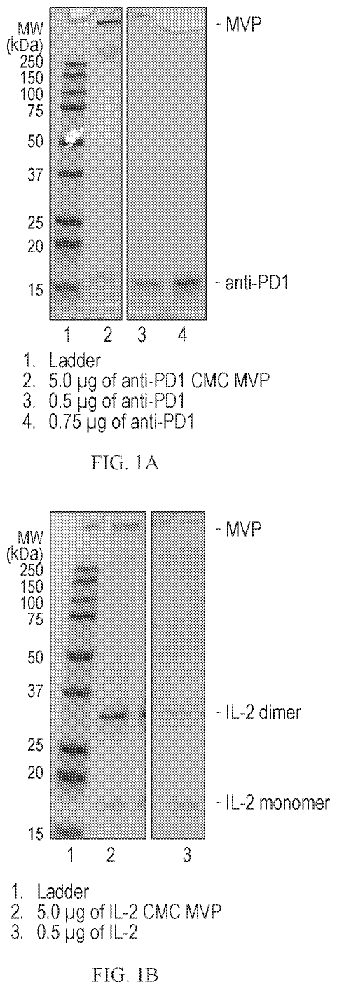

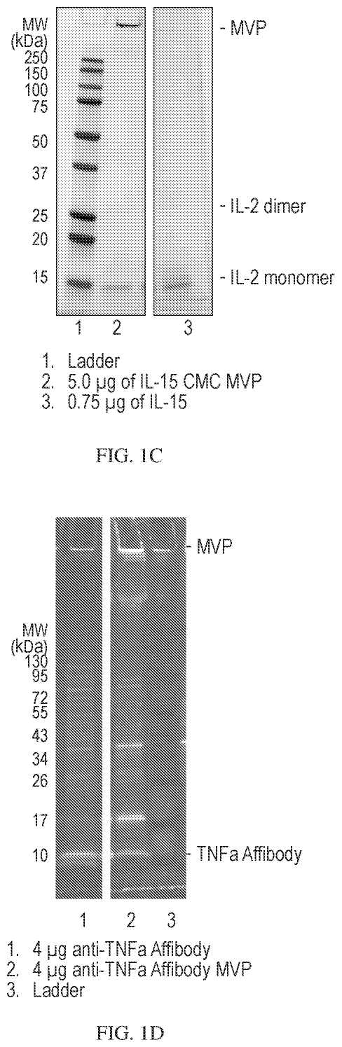

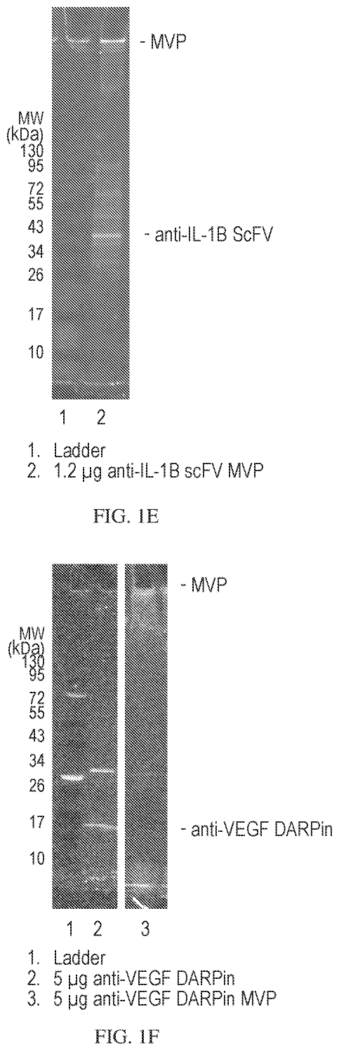

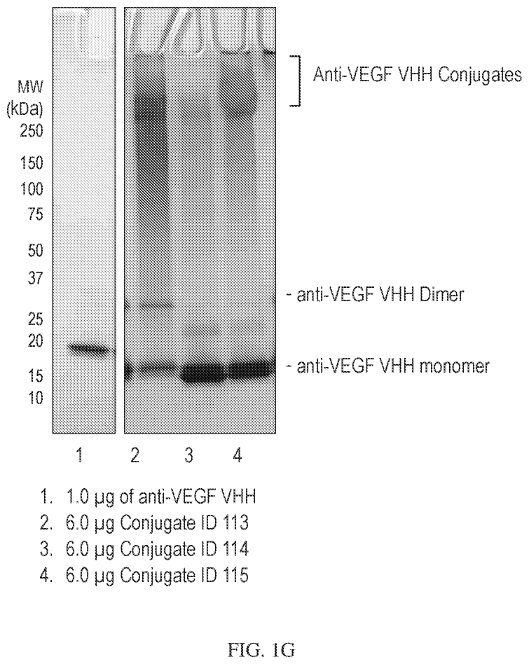

Illustrative SDS-PAGE gels of the conjugate preparations are shown in FIGS. 1A-1G.

### Binding Measurements

All reagents were equilibrated to room temperature before use for at least 30 minutes. Either one or two probes per sample (one for kinetic assay and one for ligand free control if used) were equilibrated in 250 μL BLI buffer (PBS pH 7.4, 0.2% Tween and 0.2% BSA filtered at 0.2 μm) for at minimum 10 min in a Gator Bio Max plate. Ligands were diluted to a fixed concentration of 25-100 nM based on performance in pilot reactions in BLI buffer. Analytes were prepared at the top concentration determined in pilot reactions in BLI buffer and serially diluted 1:3 two to five more times using BLI buffer (Table 3). Black flat-bottom non-coated 96 well plates (Greiner Bio One Cat #655209 or similar) were loaded column-wise with 200 μL of ligand, analyte dilutions and one column of BLI buffer for each column of ligand and analyte. One well in each column of analyte was BLI buffer to be used as a blank for reference subtraction. The sample plate was placed in the Gator on a tilted platform set to 25° C. Gator K assay loading and kinetic steps were set up using double reference and step times shown in Table 4. Ligand was loaded until signal reaches between 0.4 and 0.6 nm then returned to buffer column for a baseline measurement for 60-90 s. Next, the kinetic reads were started using the step parameters. If a ligand-free control was conducted, when kinetic reads were complete with ligand-loaded probes, the ligand free control was run using new probes that were not loaded with the ligand. The same kinetic assay timing and same sample wells were used that were analyzed with ligand loaded probes. This data was used to correct for any non-specific interactions between the sample and probe.

When kinetic assay was complete, data was analyzed using the Gator software. The raw data was corrected to include the association time after 1 second to 180 seconds. The Y-axis was aligned to the beginning of the association step and interstep correction was used. Savitzky-Goaly filtering of data was used. For single reference runs, the blank reference subtraction well was selected in the software. Then, the reference subtraction formula for each assay was edited so that for each assay it was (Kinetic Assay well-Kinetic assay buffer reference well). If a ligand-free control was conducted, the samples were set for a double reference by denoting which probes and wells were buffer references in the software. Then, the reference subtraction formula for each assay was edited so that for each assay it was a double reference with the equation of (Kinetic Assay well−Ligand Free Assay well) ˜(Kinetic Assay buffer reference well−Ligand free assay buffer well). All titrations of the same MVP were grouped by color and the parameters adjusted to a 1:1 binding model that included both association and dissociation with global, Rmax unlinked fitting. The window of interest was moved to include only 100 seconds of dissociation. The binding curve was fitted and checked that the residuals did not vary from the actual curve more than 10%, that the full R² is >0.98 and the Full X² is <3.0. The kinetics and variables K_{D}, Kₒₙ and response were noted.

TABLE 3
BLI Ligands and analyte pairings

| BLI Ligand | Tags | Analyte | Supplier | Catalog Number |
|---|---|---|---|---|
| Human VEGF | Avi | anti-VEGF | R&D Systems | AVI-293-050 |
| 165 | | | | |

TABLE 4
BLI method parameters and results specifications for kinetic quantitation

| Parameter | Wells Used | Step time (s) or info |
|---|---|---|
| Probe equilibration | Buffer in Max Plate | >600 |
| Basic Parameters | | 5 Hz, 30 s equilibration, |
| | | 1000 rpm shaking |
| Buffer | | PBS pH 7, 0.2% Tween and 0.2% |
| | | BSA filtered at 0.2 μm |
| BLI Experiment | | |
| Parameters | | |
| Baseline | Buffer Column 1 | 60 |
| Ligand loading | 100-25 nM Ligand | When loading signal is at 0.4-0.6 |
| Baseline | Buffer Column 1 | 90 |
| Association | MVP Sample(s) | 180 |
| Dissociation | Buffer Column 2 | 300 |
| Ligand free control | | |
| (with blank probes) | | |
| Baseline | Buffer Column 2 | 90 |
| Association | MVP Sample(s) | 180 |
| Dissociation | Buffer Column 2 | 300 |

TABLE 5
Properties of the purified reaction products for each peptide-polymer conjugate

| | | | Purified | | | | | |
|---|---|---|---|---|---|---|---|---|
| | | | Product | | | | | |
| | | | Peptide | | | | | |
| | | | Conc. | | | | | |
| | | | (mean ± | Percent | | | | |
| | | Peptide | SD | unconjugated | | Rh | Rh | K_{D} |
| ID | Polymer | (Seq ID) | mg/mL) | Peptide | Valency | (nm) | % PD | (pM) |
| 101 | 700 kDa | Anti- | 0.34 ± | <LOD | 13 | n.d. | n.d. | n.d. |
| | CMC | VEGF | 0.03 | | | | | |
| | | DARPin | | | | | | |
| | | (SEQ ID | | | | | | |
| | | NO: 1) | | | | | | |
| 103 | 700 kDa | Anti- | 5.24 ± | 2.4 | 76 | n.d. | n.d. | n.d. |
| | CMC | PD-1 | 0.21 | | | | | |
| | | VHH | | | | | | |
| | | (SEQ ID | | | | | | |
| | | NO: 9) | | | | | | |
| 104 | 700 kDa | IL-2 | 1.02 ± | 33.0 | 51 | 169.68 ± | 45.26 ± | n.d. |
| | CMC | (SEQ ID | 0.03 | | | 18.67 | 18.71 | |
| | | NO: 10) | | | | | | |
| 105 | 700 kDa | IL-15 | 0.99 ± | 5.0 | 49 | 68.12 ± | 53.10 ± | n.d. |
| | CMC | (SEQ ID | 0.07 | | | 9.70 | 12.98 | |
| | | NO: 11) | | | | | | |
| 106 | 700 kDa | IL- | 0.79 ± | 46.0 | 92 | 49.94 ± | 44.65 ± | n.d. |
| | CMC | 15Ra- | 0.17 | | | 3.62 | 10.79 | |
| | | IL-15 | | | | | | |
| | | fusion | | | | | | |
| | | (SEQ ID | | | | | | |
| | | NO: 12) | | | | | | |
| 107 | 700 kDa | Anti- | 0.13 ± | n.d. | 22 | n.d. | n.d. | n.d. |
| | CMC | TNFα | 0.01 | | | | | |
| | | Affibody | | | | | | |
| | | (SEQ ID | | | | | | |
| | | NO: 13) | | | | | | |
| 108 | 700 kDa | Anti-IL- | 0.04 ± | n.d. | 15 | n.d. | n.d. | n.d. |
| | CMC | 1β scFv | 0.04 | | | | | |
| | | (SEQ ID | | | | | | |
| | | NO: 14) | | | | | | |
| 109 | 830 kDa | Anti- | 0.45 ± | n.d. | 34 | n.d. | n.d. | n.d. |
| | HyA | VEGF | 0.01 | | | | | |
| | | DARPin | | | | | | |
| | | (SEQ ID | | | | | | |
| | | NO: 1) | | | | | | |
| 110 | 700 kDa | Anti- | 0.49 ± | n.d. | 20 | n.d. | n.d. | n.d. |
| | CMC | VEGF | 0.11 | | | | | |
| | | DARPin | | | | | | |
| | | (SEQ ID | | | | | | |
| | | NO: 1) | | | | | | |
| 111 | 90 kDa | Anti- | 0.86 ± | <LOD | 34 | n.d. | n.d. | n.d. |
| | CMC | VEGF | 0.02 | | | | | |
| | | DARPin | | | | | | |
| | | (SEQ ID | | | | | | |
| | | NO: 1) | | | | | | |
| 112 | 700 kDa | Anti- | 0.17 ± | n.d. | 9 | 50.45 ± | 42.96 ± | n.d. |
| | CMC | MCSP | 0.03 | | | 8.70 | 17.89 | |
| | | VHH | | | | | | |
| | | (SEQ ID | | | | | | |
| | | NO: 15) | | | | | | |
| 113 | 49 kDa | Anti- | 2.26 ± | 17.95 | 5 | n.d. | n.d. | n.d. |
| | CMC | VEGF | 0.02 | | | | | |
| | | VHH | | | | | | |
| | | (SEQ ID | | | | | | |
| | | NO: 8) | | | | | | |
| 114 | 90.5 kDa | Anti- | 2.42 ± | 48.41 | 9 | n.d. | n.d. | 966 |
| | CMC | VEGF | 0.02 | | | | | |
| | | VHH | | | | | | |
| | | (SEQ ID | | | | | | |
| | | NO: 8) | | | | | | |
| 115 | 250 kDa | Anti- | 2.71 ± | 44.37 | 21 | n.d. | n.d. | 583 |
| | CMC | VEGF | 0.01 | | | | | |
| | | VHH | | | | | | |
| | | (SEQ ID | | | | | | |
| | | NO: 8) | | | | | | |

<LOD = below limit of detection;
n.d. = not determined

### Example 2. Intratumoral Pharmacokinetic Analysis

Mouse tumor models for evaluating the clearance rate of proteins from solid tumors were used to measure the intratumoral (IT) half-life of MVPs to maximize the parameters for tumor retention. Antibodies tagged using the amine-reactive Sulfo-Cy7 NHS ester (Broadpharm Cat #BP-22541) near-infrared fluorophore by the following method were used. This method was conducted under aseptic conditions. First, the dye was dissolved DMSO at 10 mg/mL concentration. Then, the protein at 5.0-10.0 mg/mL concentration was mixed with 0.1 M sodium bicarbonate at a 3:2 vol:vol ratio. Lastly, the fluorophore was added at a 1:2 protein:fluorophore molar ratio, mixed well, and incubated at room temperature for one hour on a neutator protected from the light by covering with foil. The NHS esters were quenched by adding 1.5 M Tris buffer pH 8.5 at 10% of the reaction volume and mixed on a neutator for another 10 minutes. The tagged protein was purified away from the unreacted fluorophore using a NAP-10 desalting column (illustra Cat #17-0854-01) that was equilibrated with PBS pH 7.0+0.01% Tween-20 according to the manufacturer's directions. The protein concentration and degree of Cy7 labeling was determined by the absorbance at 280 and 750 nm. The protein as stored on ice and within 3 hours of Sulfo-Cy7 labeling, was used for MVP synthesis following the protocols described above. MVP conjugates with 90 kDa or 700 kDa CMC were generated. In each case, the final product was then sterile filtered and stored at 4° C. protected from light until used in the in vivo study.

Tumors were established by injecting 3×10⁶ SK-MEL-28 cells mixed 1:1 with Matrigel (Corning Cat #354234) in a 0.1 mL volume subcutaneously into the right flank of 8 week old athymic nude mice (Table 6). The tumor size was monitored until it reached a size of 0.1-0.2 cm. On Study Day 1, the mice with established tumors were divided into groups of 4 randomized by tumor size. Test article was removed from 4° C. storage and equilibrated to room temperature for at least 1 hour protected from light before administered by 40 μL IT injection. Mice received one of two treatments (1) Sulfo-Cy7 tagged anti-VEGF DARPin MVP with a 700 kDa CMC backbone or (2) unconjugated Sulfo-Cy7 tagged anti-VEGF DARPin, both at 300 μg anti-VEGF/mL in 40 uL IT injections. Before injection and 1, 24, 48, 72, 96, 132, and 168 hours after injection, each mouse was imaged using an in vivo imaging system (e.g. IVIS Spectrum or similar) to determine the radiant efficiency within a region of interest circumscribing the tumor compared to an untreated area on the contralateral flank. At each imaging time point, 50-μL blood samples from half of the mice in alternating cohorts were obtained to measure the drug concentration by ELISA. When the study concluded, the half-life of each treatment was determined following injection using exponential decay calculations for optical in vivo imaging (Table 7).

TABLE 6
Tumor Types Used in Xenograft Balb/c Nude Mouse Models

| Tumor Type | Cell Line | Indication |
|---|---|---|
| 1 | SK-MEL-28 | Melanoma |
| 2 | MDA-MB-231 | Triple Negative Breast Cancer |

TABLE 7
Intratumoral Half-Life of Conjugates

| | | SEQ | | | Polymer | | |
|---|---|---|---|---|---|---|---|
| | Tumor | ID | | Chemical | MW | | Intratumoral |
| Conj. | Type | NO: | Polymer | Linker | (kDa) | Valency | t_{1/2} (hrs) |
| 109 | 2 | 1 | HyA | MP2H | 830 | 34 | 20.5 ± 7.0 |
| 101 | 2 | 1 | CMC | MP2H | 700 | 13 | 101.2 ± 34.9 |
| N/A | 2 | 1 | Unconjugated | | N/A | N/A | 15.1 ± 9.8 |
| 110 | 1 | 1 | CMC | MP2H | 700 | 20.1 | 48.7 ± 24.7 |
| N/A | 1 | 1 | Unconjugated | | N/A | N/A | 8.5 ± 0.8 |
| 111 | 1 | 1 | CMC | EMCH | 90 | 34.1 ± | 18.0 ± 3.4 |
| | | | | | | 0.1 | |
| N/A | 1 | 1 | Unconjugated | | N/A | N/A | 6.5 ± 1.5 |

N/A = not applicable

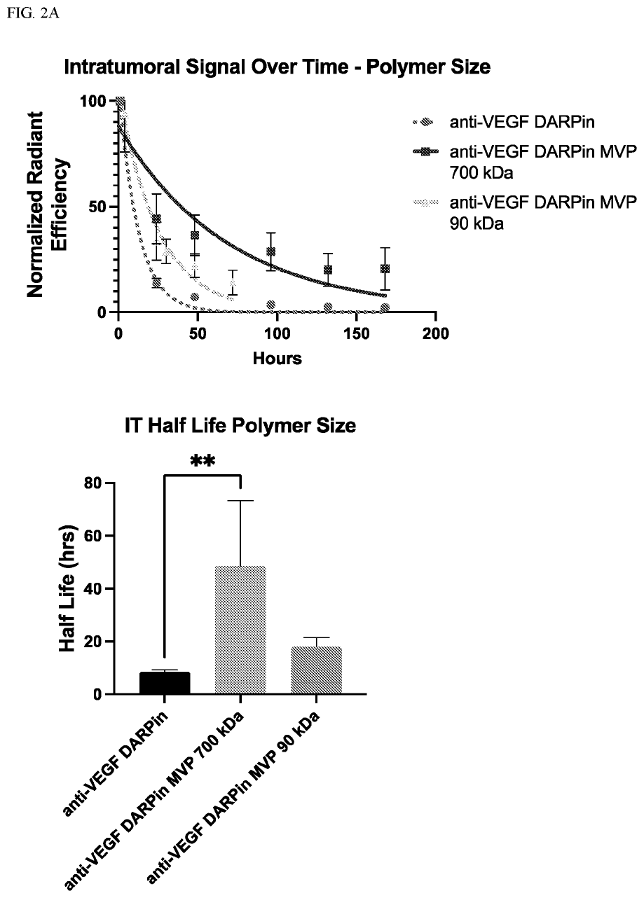

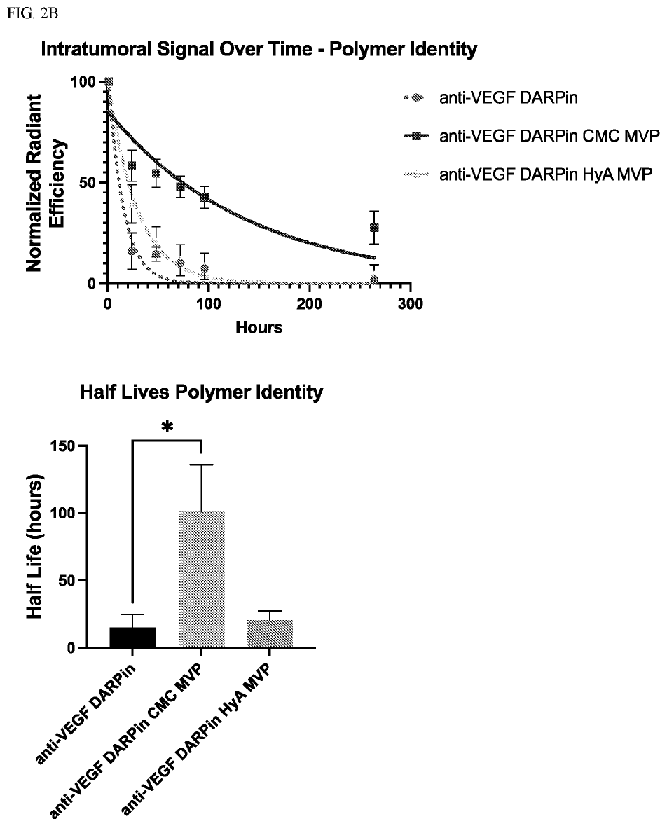

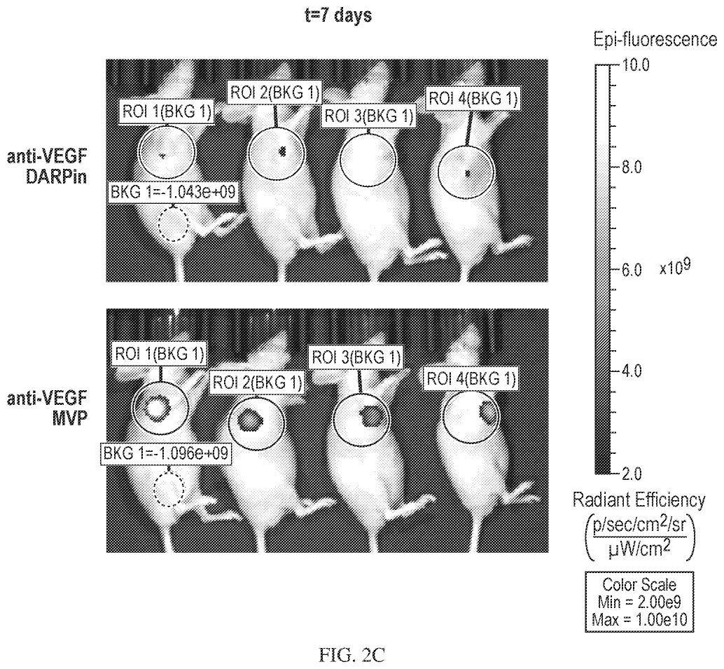

FIGS. 2A-2C show the results of experiments with an anti-VEGF MVP containing a DARPin conjugated to a 90 kDa or a 700 kDa CMC. FIG. 2A shows that the IT half-life of the MVP conjugated to a 700 kDa CMC (Conjugate 110) was longer than that of the unconjugated anti-VEGF DARPin or the peptide conjugated to a 90 kDa CMC (Conjugate 111). FIG. 2B shows that the IT half-life of the MVP conjugated to a 700 kDa CMC (Conjugate 101) was longer than that of the unconjugated anti-VEGF DARPin or the peptide conjugated to a 830 kDa hyaluronic acid (HyA) (Conjugate 109). The intratumoral half life of anti-VEGF MVPs was at least 5 times longer than the unconjugated anti-VEGF DARPin in solid tumors tested. FIG. 2C shows representative images of the mice.

### Example 3. In Vitro Bioactivity Assays

In vitro bioactivity assays are used to assess clonal expansion and cytotoxicity in co culture with tumor cells.

IL-15 MVPs that expand and activate memory and cytotoxic T cells are verified, and if necessary, any MVPs that inhibit clonal expansion of this population are eliminated using flow cytometry (Table 8). Next, three MVPs that generated the largest expansion of active cytotoxic cell populations are selected. These MVPs are added to a coculture of human TNBC cells and HLA-matched PBMCs for assessment of tumor-targeted cytotoxicity in vitro. After 6, 24, 72, and 120 hours, TNBC cell viability is quantitated using flow cytometry and/or LDH release assay. The killing efficiency of PBMCs introduced to the tumor cells that have been incubated with IL-15 MVP at 24, 48, and 72 h post addition of the MVP to the tumor cells is assessed, relative to two controls, (1) unconjugated IL-15 and (2) a vehicle, with the IL-15 concentration consistent across all groups.

TABLE 8
Cell markers for Immunophenotyping.

| Cell Type | Phenotype |
|---|---|
| CD4+ or CD8+ Tn | CD27 + CD45RA + CCR7+ |
| CD4+ or CD8+ Tcm | CD27 + CD45RA − CCR7+ |
| CD4+ or CD8+ Tem | CD27 − CD45RA − CCR7− |
| CD4+ or CD8+ TemRA | CD27 − CD45RA + CCR7− |
| CD4+ or CD8+ Tm transitional | CD27 + CD45RA − CCR7− |
| CD4+ or CD8+ Treg | CD4 + CD25 + CD127− |
| Terminally Differentiated Ts | CD3 + CD57+ |
| NK | CD3 − CD56+ |
| NK-T | CD3 + CD56 + γδTCR− |
| yδTs | gamma delta TCR+ |
| Monocytes | Scatter |
| Activated NKs | CD56 + CD335 + CD122 + (earlyCD69+) |
| Cytotoxic cell | CD25+, CD69+, CD137+, HLA-DR+ |
| activation/antigen experience | |

### Example 4. Efficacy in Melanoma Tumors

The efficacy of IL-15 MVPs to treat primary melanoma tumors is evaluated. Tumors are established by injecting SK-MEL-28 cells subcutaneously into the right flank of PBMC humanized NSG mice and tumor size is monitored until they reached 0.1-0.2 cm³. On Study Day 1, mice are divided into two treatment groups randomized by tumor size. Each group receives one of the following treatments via 40-μL IT injection: (1) IL-15 MVP with the IL-15 valency that maximizes its bioactivity in vitro, (2) unconjugated IL-15 or (3) vehicle control. The total dose of IL-15 is the same for all groups (250 μg/mL for a total of g per tumor). Twice a week until day 35, tumor size for every mouse is measured. Tumor burden and animal health are accessed daily throughout the study, euthanizing mice with tumors over 2,000 mm³ or poor health scores (BCS 2), while noting the differences in survival rate between the two groups. On days 1, 3, and 9, serum from 3 mice from each group is harvested to measure the systemic human IL-15, inflammatory cytokines, and liver enzymes in the serum using ELISA, bead capture, and colorimetric assays, respectively. On days 3 and 9, three mice are euthanized from each group to collect the tumor, spleen, and tumor draining lymph nodes. Tumor-draining lymph nodes and half of each tumor are processed for analysis using histology to quantify tumor infiltrating leukocytes (TIL) such as CD8+ T cells, NK cells, and memory T cells. On day 9 and at the study endpoint, the IT and peripheral immune cell profile are monitored by processing the other half of each tumor and spleens using flow cytometry and for a detailed quantification of local and systemic lymphocyte subpopulations.

### Example 5. Efficacy in a Breast Cancer Model

Efficacy of MVPs to treat primary breast cancer tumors and secondary metastases is validated by using an orthotopic xenograft tumor model of luciferase-expressing MDA-MB-231 TNBC cells in PBMC humanized NSG mice. A TNBC cell line from a highly metastatic tumor is selected, and the primary experimental output in these studies is quantification of lymph node, liver, and lung metastases. This study verifies that IL-15 MVPs can coordinate an anti-tumor response against human TNBC tumors the context of a human immune system.

IL-15 MVPs are synthesized using the method described above under aseptic conditions and are stored at 4° C. until use.

Orthotopic tumors are established by injecting luciferase expressing MDA-MB-231 cells into the mammary fat pads of PBMC humanized NSG mice and monitored tumor size until they reach 0.1-0.2 cm³. On Study Day 1, mice with established tumors are divided into three treatment groups randomized by tumor size (n=11: ˜50% males and ˜50% females). Each group receives one of the following treatments via 40-μL IT or 0.5-mL IV injection: (1) IL-15 MVP with the IL-15 valency that maximizes its bioactivity in vitro, (2) unconjugated IL-15 or (3) vehicle control, with the total dose of IL-15 the same for all groups (250 μg/mL for a total of 10 μg per tumor) in 40 μL IT injections.

Every 3 days until day 35, the tumor size and metastatic state of each mouse are measured using calipers and bioluminescence by injecting the animals with Luciferin and monitoring the luminescence signal with an IVIS imager (or similar instrument). The luminescence signal and primary tumor size is quantified to monitor efficacy against the primary tumor. In addition, the luminescence signal at metastases, identified as signals in the lungs and lymph nodes are also quantified. Tumor burden and animal health are accessed daily throughout the study, euthanizing mice with tumors over 2,000 mm³ or poor health scores (BCS 2) and noting survival rate differences between the groups. On days 1, 3, and 9, serum from 3 mice from each group is harvested to measure the systemic human IL-15, inflammatory cytokines, and liver enzymes in the serum using ELISA, bead capture, and colorimetric assays, respectively. On days 3 and 9, three mice from each group are euthanized, and on day 35, all remaining mice are euthanized. From each mouse, blood, liver, tumor, spleen, tumor draining lymph nodes, and lungs are collected. Tumor-draining lymph nodes and half of each tumor are processed for analysis using histology to quantify tumor infiltrating leukocytes (TIL, see Table 8 cell markers for immunotyping). Metastases are followed using bioluminescence and quantify metastasis in the lungs, lymph nodes, and liver by weight and clonogenic analysis. On day 9 and at the study endpoint, the IT and peripheral immune cell profile are monitored by processing the other half of each tumor and spleens by flow cytometry using our previously established methods.

### Example 6. Efficacy in a Syngeneic Head and Neck Cancer Model

IL-15 MVPs are synthesized using the method described above under aseptic conditions and stored at 4° C. until use. Tumors are established by injecting SCCVII cells subcutaneously in the right flank of C3H/HeJ mice and tumor size is monitored until they reach 0.1-0.2 cm³. On Study Day 1, mice with established tumors are divided into 4 groups and randomized by tumor size. Each group receives one of the following treatments via 40-μL IT or 0.5-mL IV injection: (1) IL-15 MVP conjugate the with IL-15 valency that maximizes its bioactivity in vitro, (2) unconjugated IL-15, (3) vehicle control, with the dose of total IL-15 protein the same for all treatment groups (250 μg/mL for a total of 10 μg IL-15 per tumor).

Every other day until day 25, the tumor size of six mice from each group is measured. Tumor burden and animal health are accessed daily throughout the study, euthanizing mice with tumors over 2,000 mm³ or poor health scores (BCS<2) and noting the differences in survival rate between the groups. On days 1, 3, and 9, serum from 3 mice from each group is harvested to measure the systemic human IL-15, inflammatory cytokines, and liver enzymes in the serum using ELISA, bead capture, and colorimetric assays, respectively. On days 3 and 9, three mice from each group are euthanized. From each mouse, the tumor, spleen and tumor draining lymph nodes are collected. The tumor-draining lymph nodes and half of each tumor are processed for analysis using immunohistochemistry to quantify activated TILs, particularly cytotoxic CD8+ T cells, NKp46+NK cells, that express activation and proliferation markers. On day 9 and at the study endpoint, the IT and peripheral immune cell profile are monitored by processing the other half of each tumor and spleens by flow cytometry using our previously established methods.

### Example 7. Systemic Pharmacokinetic Analysis

Mouse models were used to evaluating the biodistribution of proteins after intravenous administration. In some cases, proteins were tagged with an ¹²⁵I radiolabel using methods that are well established to those skilled in the art such as using a ¹²⁵I Bolton-Hunter reagent. Tumors were established by injecting 3×10⁶ tumor cells mixed 1:1 with Matrigel (Corning Cat #354234) in a 0.1 mL volume subcutaneously into the right flank of 8 week old athymic nude mice (Table 6). The tumor size was monitored until it reached a size of 0.1-0.2 cm. On Study Day 1, the mice with established tumors were divided into groups randomized by tumor size. Mice received a vehicle control or treatments consisting of anti-VEGF MVP with CMC backbone of various sizes or unconjugated anti-VEGF proteins. All treatments were 300 μg anti-VEGF/mL in 0.5 mL IV injections. Before injection and 1, 24, 48, 72, 96, 132, and 168 hours after injection, each 3 mice from each group were euthanized and tissues were collected for biodistribution analysis, including from the tumor, blood, heart, lungs, brain, kidneys, liver, small intestines, large intestines, adrenal gland, thymus, ovaries, testes, bladder, muscle, and fat. The concentration of the treatment in each tissue was measured using one of the following methods: ELISA using antibodies raised against the test article, LC/MS after tryptic digestion, or radiolabel detection.

TABLE 9
Sequences

aVEGF_Gly_Cys DARPin (SEQ ID NO: 1)

| | |
|---|---|
| SNAGSDLDKK LLEAARAGQD DEVRILMANG ADVNARDSTG WTPLHLAAPW | 50 |
| GHPEIVEVLL KNGADVNAAD FQGWTPLHLA AAVGHLEIVE VLLKYGADVN | 100 |
| AQDKFGKTAF DISIDNGNED LAEILQKAAG GGSGGGSC | |

IL-15 (SEQ ID NO: 2)

| | |
|---|---|
| SNANWVNVIS DLKKIEDLIQ SMHIDATLYT ESDVHPSCKV TAMKCFLLEL | 50 |
| QVISLESGDA SIHDTVENLI ILANNSLSSN GNVTESGCKE CEELEEKNIK | 100 |
| EFLQSFVHIV QMFINTS | |

Anti-VEGF VHH (SEQ ID NO: 3)

| | |
|---|---|
| MQVQLVESGG GLVQPGGSLR LSCAASGFAY STYSMGWFRQ APGKEREAVA | 50 |
| TINSGTFRLW YTDSVKGRFT ISRDNSKNTL YLQMNSLRPE DTAVYYCAAR | 100 |
| AWSPYSSTVD AGDFRYWGQG TLVTVSS | |

Anti-PD-1 VHH (SEQ ID NO: 4)

| | |
|---|---|
| SNAEVQLVES GGGLVQPGGS LRLSCAASGS IFSIHAMGWF RQAPGKEREF | 50 |
| VAAITWSGGI TYYEDSVKGR FTISRDNSKN TVYLQMNSLR PEDTAVYYCA | 100 |
| ADRAESSWYD YWGQGTLVTV SS | |

IL-2 (SEQ ID NO: 5)

| | |
|---|---|
| SNAAPTSSST KKTQLQLEHL LLDLQMILNG INNYKNPKLT RMLTFKFYMP | 50 |
| KKATELKHLQ CLEEELKPLE EVLNLAQSKN FHLRPRDLIS NINVIVLELK | 100 |
| GSETTFMCEY ADETATIVEF LNRWITFSQS IISTLT | |

IL-15Ra-IL-15 fusion (SEQ ID NO: 6)

| | |
|---|---|
| SNAGITCPPP MSVEHADIWV KSYSLYSRER YICNSGFKRK AGTSSLTECV | 50 |
| LNKATNVAHW TTPSLKCIRS GGSGGGGSGG GSGGGGSLQN WVNVISDLKK | 100 |
| IEDLIQSMHI DATLYTESDV HPSCKVTAMK CELLELQVIS LESGDASIHD | 150 |
| TVENLIILAN NSLSSNGNVT ESGCKECEEL EEKNIKEFLQ SFVHIVQMFI | 200 |
| NTS | |

Anti-IL-1B scFv (SEQ ID NO: 7)

| | |
|---|---|
| SNAEIVMTQS PSTLSASVGD RVIITCQASQ SIDNWLSWYQ QKPGKAPKLL | 50 |
| IYRASTLASG VPSRFSGSGS GAEFTLTISS LQPDDFATYY CQNTGGGVSI | 100 |
| AFGQGTKLTV LGGGGGSGGG GSGGGGSGGG GSEVQLVESG GGLVQPGGSL | 150 |
| RLSCTASGFS LSSAAMAWVR QAPGKGLEWV GIIYDSASTY YASWAKGRFT | 200 |
| ISRDTSKNTV YLQMNSLRAE DTAVYYCARE RAIFSGDFVL WGQGTLVTVS | 250 |
| S | |

Anti-VEGF VHH (SEQ ID NO: 8)

| | |
|---|---|
| MQVQLVESGG GLVQPGGSLR LSCAASGFAY STYSMGWERQ APGKEREAVA | 50 |
| TINSGTFRLW YTDSVKGRFT ISRDNSKNTL YLQMNSLRPE DTAVYYCAAR | 100 |
| AWSPYSSTVD AGDFRYWGOG TLVTVSSAEA AAKEAAAKEA AAKAGC | 146 |

Anti-PD-1 VHH (SEQ ID NO: 9)

| | |
|---|---|
| SNAEVQLVES GGGLVQPGGS LRLSCAASGS IFSIHAMGWF RQAPGKEREF | 50 |
| VAAITWSGGI TYYEDSVKGR FTISRDNSKN TVYLQMNSLR PEDTAVYYCA | 100 |
| ADRAESSWYD YWGQGTLVTV SSAEAAAKEA AAKEAAAKAG C | 141 |

IL-2 (SEQ ID NO: 10)

| | |
|---|---|
| SNAAPTSSST KKTQLQLEHL LLDLQMILNG INNYKNPKLT RMLTFKFYMP | 50 |
| KKATELKHLQ CLEEELKPLE EVLNLAQSKN FHLRPRDLIS NINVIVLELK | 100 |
| GSETTFMCEY ADETATIVEF LNRWITFSQS IISTLTAEAA AKEAAAKEAA | 150 |
| AKAGC | 155 |

IL-15 (SEQ ID NO: 11)

| | |
|---|---|
| SNANWVNVIS DLKKIEDLIQ SMHIDATLYT ESDVHPSCKV TAMKCFLLEL | 50 |
| QVISLESGDA SIHDTVENLI ILANDSLSSN GNVTESGCKE CEELEEKNIK | 100 |
| EFLQSFVHIV QMFINTSAAE AAAKEAAAKE AAAKAGC | 137 |

IL-15Ra-IL-15 fusion (SEQ ID NO: 12)

| | |
|---|---|
| SNAGITCPPP MSVEHADIWV KSYSLYSRER YICNSGFKRK AGTSSLTECV | 50 |
| LNKATNVAHW TTPSLKCIRS GGSGGGGSGG GSGGGGSLQN WVNVISDLKK | 100 |
| IEDLIQSMHI DATLYTESDV HPSCKVTAMK CFLLELQVIS LESGDASIHD | 150 |
| TVENLIILAN NSLSSNGNVT ESGCKECEEL EEKNIKEFLQ SFVHIVQMFI | 200 |
| NTSAAEAAAK EAAAKEAAAK AGC | 223 |

Anti-TNFa Affibody (SEQ ID NO: 13)

| | |
|---|---|
| CGGGVDNKFN KEVGWAFGEI GALPNLNALQ FRAFIISLWD DPSQSANLLA | 50 |
| EAKKLNDAQA PK | 62 |

Anti-IL-1B scFv (SEQ ID NO: 14)

| | |
|---|---|
| SNAEIVMTQS PSTLSASVGD RVIITCQASQ SIDNWLSWYQ QKPGKAPKLL | 50 |
| IYRASTLASG VPSRFSGSGS GAEFTLTISS LQPDDFATYY CQNTGGGVSI | 100 |
| AFGQGTKLTV LGGGGGSGGG GSGGGGSGGG GSEVQLVESG GGLVQPGGSL | 150 |
| RLSCTASGFS LSSAAMAWVR QAPGKGLEWV GIIYDSASTY YASWAKGRFT | 200 |
| ISRDTSKNTV YLQMNSLRAE DTAVYYCARE RAIFSGDFVL WGQGTLVTVS | 250 |
| SSPSTPPTPS PSTPPGGC | 268 |

Anti-MCSP VHH (SEQ ID NO: 15)

| | |
|---|---|
| SNAEVQLQAS GGGFVQPGGS LRLSCAASGT YSRITTMGWF RQAPGKEREF | 50 |
| VSAISFASDN TPYYADSVKG RFTISRDNSK NTVYLQMNSL RAEDTATYYC | 100 |
| AAWASRSTKA PMRYWGQGTQ VTVSSAEAAA KEAAAKEAAA KAGC | 144 |

Anti-MCSP VHH (SEQ ID NO: 16)

| | |
|---|---|
| SNAEVQLQAS GGGFVQPGGS LRLSCAASGT YSRITTMGWF RQAPGKEREF | 50 |
| VSAISFASDN TPYYADSVKG RFTISRDNSK NTVYLQMNSL RAEDTATYYC | 100 |
| AAWASRSTKA PMRYWGQGTQ VTVSS | 125 |

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A conjugate of Formula (I):
(X—Y)ₙ—Z (I),
wherein
each X is a peptide having a molecular weight of from about 5 kDa to about 150 kDa;
each Y is an organic linker having a molecular weight of from about 100 Da to about 500 Da;
Z is a biocompatible cellulose polymer having a molecular weight of from about 0.01 MDa to about 3 MDa; and
subscript n is an integer of from about 5 to about 500.

2. The conjugate of claim 1, wherein
each X is a peptide having a molecular weight of from about 5 kDa to about 150 kDa;
each Y is an organic linker having a molecular weight of from about 100 Da to about 500 Da;
Z is a biocompatible cellulose polymer having a molecular weight of from about 0.1 MDa to about 3 MDa; and
subscript n is an integer of from about 5 to about 500.

3. The conjugate of claim 1, wherein the peptide modulates the activity of immune cell function.

4. The conjugate of claim 1 or 3, wherein the peptide selectively binds B cell maturation antigen (BCMA), CD19, CD20, CD40, CD44, CD47, CD80, cytotoxic T-lymphocyte-associated antigen 4 (CTLA4), epidermal growth factor receptor (EGFR), epithelial cell adhesion molecule (EpCAM), glucose transport protein (GTR), human epithelial growth factor receptor 2 (HER2), interleukin-5 (IL-5), interleukin-18 (IL-18), melanoma-associated chondroitin sulfate proteoglycan (MCSP), mesothelin, programmed cell death protein 1 (PD-1), programmed death-ligand 1 (PD-L1), programmed cell death 1 ligand 2 (PD-L2), tumor necrosis factor-alpha (TNF-α), unique long-16 binding protein 2 (ULBP2), unique long-16 binding protein 5 (ULBP5), unique long-16 binding protein 6 (ULBP6), or vascular endothelial growth factor receptor (VEGFR).

5. The conjugate of any one of claims 1 to 4, wherein the peptide selectively binds epidermal growth factor receptor (EGFR), tumor necrosis factor-alpha (TNF-α), or vascular endothelial growth factor receptor (VEGFR).

6. The conjugate of claim 1 or 3, wherein the peptide selectively binds interleukin-2 (IL-2) or interleukin-15 (IL-15).

7. The conjugate of any one of claims 1 to 6, wherein the peptide is a monoclonal IgG, an IgG fragment, single chain scFv, single-domain heavy-chain VHH, adnectin, affibody, anticalin, DARPin, or an engineered Kunitz-type inhibitor.

8. The conjugate of any one of claims 1 to 7, wherein the peptide has an amino acid sequence according to any one of SEQ ID NOs: 1 to 15.

9. The conjugate of any one of claims 1 to 8, wherein the organic linker comprises a succinimide.

10. The conjugate of any one of claims 1 to 9, wherein the organic linker has the structure:

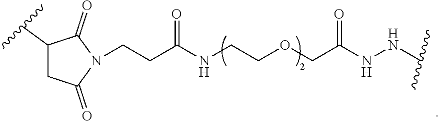

11. The conjugate of any one of claims 1 to 10, wherein the biocompatible cellulose polymer comprises carboxymethyl cellulose.

12. The conjugate of any one of claims 1 to 11, wherein the biocompatible cellulose polymer has a molecular weight of from about 0.1 MDa to about 1 MDa.

13. The conjugate of any one of claims 1 to 12, wherein the biocompatible cellulose polymer has a molecular weight of about 0.7 MDa.

14. The conjugate of any one of claims 1 to 13, wherein subscript n is an integer of from about 5 to about 200.

15. The conjugate of any one of claims 1 to 14, wherein subscript n is an integer of from about 10 to about 120.

16. The conjugate of any one of claims 1 to 15, having an in vivo half-life of at least 5 times longer than an unconjugated peptide.

17. A conjugate of Formula (Ia):
(X—Y)ₙ—Z (Ia),
wherein
X is a peptide having an amino acid sequence of SEQ ID NO: 1;
Y is an organic linker having the structure: 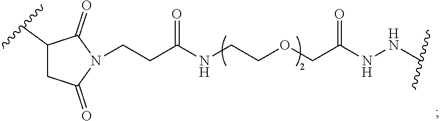
Z is a carboxymethyl cellulose polymer having a molecular weight of about 0.7 MDa; and
subscript n is an integer of from about 10 to about 50.

18. A pharmaceutical composition comprising a conjugate of any one of claims 1 to 17, and a pharmaceutically acceptable excipient.

19. A method of treating a cancer in a human subject in need thereof, comprising administering to the subject a therapeutically effective amount of a conjugate of any one of claims 1 to 17, or a pharmaceutical composition of claim 18.

20. The method of claim 19, wherein the cancer is a solid tumor.

21. The method of claim 19 or 20, wherein the cancer is a bladder cancer, bone cancer, brain cancer, breast cancer, cervical cancer, central nervous system (CNS) cancer, colorectal cancer, endometrial cancer, esophageal cancer, gastric cancer, head and neck cancer, hepatocellular cancer, lung cancer, lymphoma, melanoma, neuroendocrine cancer, ovarian cancer, pancreatic cancer, prostate cancer, renal cancer, sarcoma, or thyroid cancer.

22. The method of claim 21, wherein the cancer is a melanoma.

23. The method of claim 21, wherein the cancer is a head and neck squamous cell carcinoma.

24. The method of claim 21, wherein the cancer is a triple-negative breast cancer.

25. The method of any one of claims 19 to 24, wherein the conjugate is administered intratumorally.
